SEQUENCE LISTING (13 sequences)

SEQ ID NO: 1 - DNA, 25 bp, synthetic construct
  misc_feature 1..25: note = HOXB13 forward primer
  source 1..25: mol_type = other DNA
gattaccatctggtttcagaaccgc

SEQ ID NO: 2 - DNA, 28 bp, synthetic construct
  misc_feature 1..28: note = HOXB13 reverse primer
  source 1..28: mol_type = other DNA
gctcaattcatgaaagcggtttctaaag

SEQ ID NO: 3 - DNA, 31 bp, synthetic construct
  misc_feature 1..31: note = Mutagenesis forward primer
  source 1..31: mol_type = other DNA
gcgctacccctaagagatctccttgcctggg

SEQ ID NO: 4 - DNA, 31 bp, synthetic construct
  misc_feature 1..31: note = Mutagenesis reverse primer
  source 1..31: mol_type = other DNA
ggagatctcttaggggtagcgctgttcttca

SEQ ID NO: 5 - DNA, 22 bp, synthetic construct
  misc_feature 1..22: note = HOXB13 qRT-PCT forward primer
  source 1..22: mol_type = other DNA
gaacagccagatgtgttgccag

SEQ ID NO: 6 - DNA, 20 bp, synthetic construct
  misc_feature 1..20: note = HOXB13 qRT-PCR reverse primer
  source 1..20: mol_type = other DNA
ggaatgcgtttcttgcggcc

SEQ ID NO: 7 - DNA, 21 bp, synthetic construct
  misc_feature 1..21: note = CCNB1 qRT-PCR forward primer
  source 1..21: mol_type = other DNA
ctgagccagaacctgagcctg

SEQ ID NO: 8 - DNA, 23 bp, synthetic construct
  misc_feature 1..23: note = CCNB1 qRT-PCR reverse primer
  source 1..23: mol_type = other DNA
gtcttcttctgcaggggcacatc

SEQ ID NO: 9 - DNA, 19 bp, synthetic construct
  misc_feature 1..19: note = GAPDH qRT-PCR forward primer
  source 1..19: mol_type = other DNA
agcaccaggtggtctcctc

SEQ ID NO: 10 - DNA, 22 bp, synthetic construct
  misc_feature 1..22: note = GAPDH qRT-PCR reverse primer
  source 1..22: mol_type = other DNA
ccctgttgctgtagccaaattc

SEQ ID NO: 11 - protein, 284 aa, Homo sapiens
MEPGNYATLDGAKDIEGLLGAGGGRNLVAHSPLTSHPAAPTLMPAVNYAPLDLPGSAEPPKQCHPCPGVPQGTSPAPVPYGYFGGGYYSCRVSRSSLKPCAQAATLAAYPAETPTAGEEYPSRPTEFAFYPGYPGTYQPMASYLDVSVVQTLGAPGEPRHDSLLPVDSYQSWALAGGWNSQMCCQGEQNPPGPFWKAAFADSSGQHPPDACAFRRGRKKRIPYSKGQLRELEREYAANKFITKDKRRKISAATSLSERQITIWFQNRRVKEKKVLAKVKNSATP

SEQ ID NO: 12 - DNA, 3039 bp, Homo sapiens
  source 1..3039: mol_type = other DNA
ctcttgcgtcaagacggccgtgctgagcgaatgcaggcgacttgcgagctgggagcgatttaaaacgctttggattcccccggcctgggtggggagagcgagctgggtgccccctagattccccgcccccgcacctcatgagccgaccctcggctccatggagcccggcaattatgccaccttggatggagccaaggatatcgaaggcttgctgggagcgggaggggggcggaatctggtcgcccactcccctctgaccagccacccagcggcgcctacgctgatgcctgctgtcaactatgcccccttggatctgccaggctcggcggagccgccaaagcaatgccacccatgccctggggtgccccaggggacgtccccagctcccgtgccttatggttactttggaggcgggtactactcctgccgagtgtcccggagctcgctgaaaccctgtgcccaggcagccaccctggccgcgtaccccgcggagactcccacggccggggaagagtaccccagccgccccactgagtttgccttctatccgggatatccgggaacctaccagcctatggccagttacctggacgtgtctgtggtgcagactctgggtgctcctggagaaccgcgacatgactccctgttgcctgtggacagttaccagtcttgggctctcgctggtggctggaacagccagatgtgttgccagggagaacagaacccaccaggtcccttttggaaggcagcatttgcagactccagcgggcagcaccctcctgacgcctgcgcctttcgtcgcggccgcaagaaacgcattccgtacagcaaggggcagttgcgggagctggagcgggagtatgcggctaacaagttcatcaccaaggacaagaggcgcaagatctcggcagccaccagcctctcggagcgccagattaccatctggtttcagaaccgccgggtcaaagagaagaaggttctcgccaaggtgaagaacagcgctaccccttaagagatctccttgcctgggtgggaggagcgaaagtgggggtgtcctggggagaccaggaacctgccaagcccaggctggggccaaggactctgctgagaggcccctagagacaacacccttcccaggccactggctgctggactgttcctcaggagcggcctgggtacccagtatgtgcagggagacggaaccccatgtgacagcccactccaccagggttcccaaagaacctggcccagtcataatcattcatcctgacagtggcaataatcacgataaccagtactagctgccatgatcgttagcctcatattttctatctagagctctgtagagcactttagaaaccgctttcatgaattgagctaattatgaataaatttggaaggcgatccctttgcagggaagctttctctcagacccccttccattacacctctcaccctggtaacagcaggaagactgaggagaggggaacgggcagattcgttgtgtggctgtgatgtccgtttagcatttttctcagctgacagctgggtaggtggacaattgtagaggctgtctcttcctccctccttgtccaccccatagggtgtacccactggtcttggaagcacccatccttaatacgatgatttttctgtcgtgtgaaaatgaagccagcaggctgcccctagtcagtccttccttccagagaaaaagagatttgagaaagtgcctgggtaattcaccattaatttcctcccccaaactctctgagtcttcccttaatatttctggtggttctgaccaaagcaggtcatggtttgttgagcatttgggatcccagtgaagtagatgtttgtagccttgcatacttagcccttcccaggcacaaacggagtggcagagtggtgccaaccctgttttcccagtccacgtagacagattcacagtgcggaattctggaagctggagacagacgggctctttgcagagccgggactctgagagggacatgagggcctctgcctctgtgttcattctctgatgtcctgtacctgggctcagtgcccggtgggactcatctcctggccgcgcagcaaagccagcgggttcgtgctggtccttcctgcaccttaggctgggggtggggggcctgccggcgcattctccacgattgagcgcacaggcctgaagtctggacaacccgcagaaccgaagctccgagcagcgggtcggtggcgagtagtggggtcggtggcgagcagttggtggtgggccgcggccgccactacctcgaggacatttccctcccggagccagctctcctagaaaccccgcggcggccgccgcagccaagtgtttatggcccgcggtcgggtgggatcctagccctgtctcctctcctgggaaggagtgagggtgggacgtgacttagacacctacaaatctatttaccaaagaggagcccgggactgagggaaaaggccaaagagtgtgagtgcatgcggactgggggttcaggggaagaggacgaggaggaggaagatgaggtcgatttcctgatttaaaaaatcgtccaagccccgtggtccagcttaaggtcctcggttacatgcgccgctcagagcaggtcactttctgccttccacgtcctccttcaaggaagccccatgtgggtagctttcaatatcgcaggttcttactcctctgcctctataagctcaaacccaccaacgatcgggcaagtaaaccccctccctcgccgacttcggaactggcgagagttcagcgcagatgggcctgtggggagggggcaagatagatgagggggagcggcatggtgcggggtgaccccttggagagaggaaaaaggccacaagaggggctgccaccgccactaacggagatggccctggtagagacctttgggggtctggaacctctggactccccatgctctaactcccacactctgctatcagaaacttaaacttgaggattttctctgtttttcactcgcaataaattcagagcaaacaaaa

SEQ ID NO: 13 - protein, 380 aa, Homo sapiens
MEPGNYATLDGAKDIEGLLGAGGGRNLVAHSPLTSHPAAPTLMPAVNYAPLDLPGSAEPPKQCHPCPGVPQGTSPAPVPYGYFGGGYYSCRVSRSSLKPCAQAATLAAYPAETPTAGEEYPSRPTEFAFYPGYPGTYQPMASYLDVSVVQTLGAPGEPRHDSLLPVDSYQSWALAGGWNSQMCCQGEQNPPGPFWKAAFADSSGQHPPDACAFRRGRKKRIPYSKGQLRELEREYAANKFITKDKRRKISAATSLSERQITIWFQNRRVKEKKVLAKVKNSATPKRSPCLGGRSESGGVLGRPGTCQAQAGAKDSAERPLETTPFPGHWLLDCSSGAAWVPSMCRETEPHVTAHSTRVPKEPGPVIIIHPDSGNNHDNQY

(19) United States
(12) Patent Application Publication
(10) Pub. No.: US 2026/0193714 A1
(43) Pub. Date: Jul. 9, 2026

(54) A GENETIC VARIANT, X285K IN HOXB13, IS ASSOCIATED WITH RISK OF AGGRESSIVE PROSTATE CANCER IN MEN OF AFRICAN ANCESTRY

(71) Applicant: The Johns Hopkins University, Baltimore, MD (US)

(72) Inventors: William Brewster Isaacs, Glyndon, MD (US); Jun Luo, Clarksville, MD (US); Mayuko Kanayama, Baltimore, MD (US)

(21) Appl. No.: 19/129,469

(22) PCT Filed: Oct. 3, 2023
(86) PCT No.: PCT/US2023/075775

§ 371 Date: May 13, 2025

Related U.S. Application Data
(60) Provisional application No. 63/383,592, filed on Nov. 14, 2022.

(51) Int. Cl. C12Q 1/6886; G01N 33/57555

(52) U.S. Cl. CPC C12Q 1/6886; G01N 33/57555; C12Q 2600/106; C12Q 2600/118; C12Q 2600/156

(57) ABSTRACT

The present invention relates to the field of cancer. More specifically, the present invention provides compositions and methods useful for treating patients having a HOXB13 genetic variant. In a specific embodiment, a method for identifying a subject as having an increased risk of prostate cancer (PCa) comprises the step of detecting single nucleotide polymorphism rs77179853 in DNA isolated from the subject, wherein the presence of the rs77179853 SNP identifies the subject as having an increased risk of aggressive prostate cancer.

### CROSS-REFERENCE TO RELATED APPLICATIONS

This application claims the benefit of U.S. Provisional Application No. 63/383,592, filed Nov. 14, 2022, which is incorporated herein by reference in its entirety.

### FIELD OF THE INVENTION

The present invention relates to the field of cancer. More specifically, the present invention provides compositions and methods useful for treating patients having a HOXB13 genetic variant.

### REFERENCE TO AN ELECTRONIC SEQUENCE LISTING

The text of the computer readable sequence listing filed herewith, titled “P17235-02”, created Sep. 25, 2023, having a file size of 19,903 bytes, is hereby incorporated by reference in its entirety.

### BACKGROUND OF THE INVENTION

African-American (AA) men are more likely to be diagnosed with advanced prostate cancer (PCa) and are nearly twice as likely to die from the disease than men of European ancestry. While the reasons are complex and not fully understood, this disparity cannot be fully accounted for by differences in access to care or socio-economic status, indicating that there are biological contributions.

While numerous studies in predominantly European-American patients have leveraged large cohorts to identify the molecular drivers of PCa, few equivalent studies have been conducted in patients of African ancestry. For example, the Cancer Genome Atlas (TCGA) sequencing effort for PCa is minimally informative for AA, as it contains only 14% (n=43) AA men, of whom only 17 have the intermediate or high-risk disease. These low numbers, combined with extensive disease heterogeneity, have hampered discovery efforts to identify genomic drivers of PCa among AA populations. Thus, the biological determinants of PCa risk overall and aggressive disease in particular in AA men are still unclear and their discovery remains a critical and unmet need in cancer health disparities research. The present inventors' lack of knowledge about the molecular drivers of PCa among AA men remains a major barrier to the implementation of precision medicine in this high-risk population.

The identification of the first bona fide PCa-specific susceptibility gene, HOXB13, a prostate-specific transcription factor, was reported in 2012. Using positional information from linkage analyses in PCa families, a rare but recurrent missense change, G84E, was identified in the HOXB13 gene on 17q21. In an analysis of germline DNA from over 5000 PCa cases and controls, the frequency of G84E was significantly higher in cases (1.4%) than controls (0.1-0.4%). An enrichment of G84E was found in PCa patients who were diagnosed at an early age and with a positive family history of PCa. These findings have been consistently confirmed, with odds ratio (OR) s for PCa varying from 2- to 15-fold. Combined analyses of different study populations in the International Consortium for Prostate Cancer Genetics demonstrated that the most common variant in HOXB13 in US men, G84E, had the highest frequency in individuals of Nordic descent. Indeed, as many as 8-10% of Swedish and Finnish men with family history-positive PCa diagnosed at an early age carry a G84E HOXB13 variant, compared to ~1% or less in unaffected men in these populations. A critical additional finding was that all G84E variant carriers shared a common haplotype, i.e., they are all descended from a common founder, presumably of Swedish or Finnish origin. Despite the established role of HOXB13 G84E in PCa susceptibility in men of European descent, little is known about a possible role for HOXB13 variants in PCa risk in men of African ancestry.

The HOXB13 gene is abundantly and highly specifically expressed in cells of the prostate lineage including both benign and malignant epithelium, and its expression is typically maintained in late-stage metastatic prostate cancers with aggressive treatment-resistant phenotypes. HOXB13 interacts with the androgen receptor (AR) and is a bifunctional regulator of AR target genes, acting as both a suppressor and an activator of androgen-responsive genes. Multiple genetic variants associated with prostate cancer risk have been found in the HOXB13 gene in different ancestral populations including European, Japanese, and Chinese. However, none of these variants are preferentially associated with risk for aggressive prostate cancer. For example, the most common HOXB13 variant in US men of predominantly European ancestry, G84E, was reproducibly associated with risk for prostate cancer, but the association was equally strong in men with aggressive and non-aggressive disease. As such, though existing pathogenic HOXB13 mutations can be incorporated in genetic testing to enable targeted screening6, they lack prognostic and treatment utility.

### SUMMARY OF THE INVENTION

The present invention is based, at least in part, on the discovery that the HOXB13 variant (X285K) mediates an aggressive phenotype in prostate cancer in African-American (AA) men. While this variant is rare in the AA population (~0.2% MAF), its ancestry-specific occurrence and apparent preferential association with risk for the more aggressive disease at an early age emphasizes its translational potential as an important, novel PCa susceptibility marker in the high-risk AA population.

In one aspect, the present invention provides methods for identifying a subject as having an increased risk of prostate cancer (PCa). In one embodiment, a method comprises the step of detecting single nucleotide polymorphism rs77179853 in DNA isolated from the subject, wherein the presence of the rs77179853 SNP identifies the subject as having an increased risk of aggressive prostate cancer. In specific embodiments, the PCa is aggressive PCa. In other embodiments, the subject is of African descent.

In another embodiment, the subject has a family history of PCa. In a further embodiment, the subject is identified as having an increased risk of PCa at an early age. In yet another embodiment, the DNA is isolated from a biological sample selected from the group consisting of prostate tissue biopsy, fresh or archival surgical specimen, saliva, urine and blood. The methods of the present invention can further comprise the step of administering a treatment of prostatectomy, radiation, chemotherapy, immunotherapy or a combination thereof to the subject having an increased risk of PCa.

In specific embodiments, the detecting step comprises nucleic acid amplification. In another embodiment, the detecting step comprises a hybridization reaction. In further embodiments, the hybridization reaction further comprises hybridizing the sample to one or more primer sets. In specific embodiments, the primer sets amplify one or more nucleic acids that encode amino acids 285-380 of SEQ ID NO: 13.

In one embodiment, the hybridization reaction is a polymerase chain reaction (PCR). In another embodiment, the PCR is reverse transcription PCR. In the methods of the present invention, the detecting step comprises a microarray or DNA sequencing.

In another aspect, the present invention provides methods for determining whether a subject will not respond to androgen or androgen receptor-directed therapies. In certain embodiments, the method comprises the step of detecting single nucleotide polymorphism rs77179853 in DNA isolated from the subject, wherein the presence of the rs77179853 SNP indicates that the subject will not respond, or have poor outcome, to androgen or androgen receptor-directed therapies.

In another aspect, the present invention provides methods treating a subject at risk of or having PCa. In a specific embodiment, a method comprises the steps of (a) detecting single nucleotide polymorphism rs77179853 in DNA isolated from the subject, wherein the presence of the rs77179853 SNP identifies the subject as having poor response to androgen or androgen receptor-directed therapies; and (b) administering a non-androgen or non-androgen receptor therapies to treat the subject. In particular embodiments, the non-androgen or non-androgen receptor therapies comprises prostatectomy, radiation, chemotherapy, immunotherapy or a combination thereof.

In other embodiments, a method for identifying a subject as having an increased risk of prostate cancer (PCa) comprises the step of detecting a protein comprising SEQ ID NO:13 or a nucleic acid encoding SEQ ID NO:13 in a sample obtained from the subject, wherein the presence of the protein comprising SEQ ID NO: 13 or the nucleic acid encoding SEQ ID NO:13 identifies the subject as having an increased risk of aggressive prostate cancer.

In certain embodiments, a method for determining whether a subject will not respond to androgen therapy comprises the step of detecting a protein comprising SEQ ID NO:13 or a nucleic acid encoding SEQ ID NO:13 in a sample obtained from the subject, wherein the presence of the protein comprising SEQ ID NO: 13 or the nucleic acid encoding SEQ ID NO:13 indicates that the subject will not respond to androgen therapy

In such embodiments, amino acid 285 of SEQ ID NO:13 is a lysine. In a particular embodiment, detecting a protein comprising SEQ ID NO:13 or a nucleic acid encoding SEQ ID NO: 13 comprises detecting one or more of amino acids 285-380 of SEQ ID NO:13. In ore particular embodiments, detecting a protein comprising SEQ ID NO:13 or a nucleic acid encoding SEQ ID NO:13 comprises detecting one or more of amino acids 344-364 of SEQ ID NO: 13. In specific embodiments, the subject is of African descent.

### BRIEF DESCRIPTION OF THE FIGURES

FIG. 1A-1B. FIG. 1A: Schematic diagram of HOXB13 bidirectional inducible vectors in stable LNCaP95 clones including 5 LN95WT, 3 LNX285K, and 3 LN95NT clones. FIG. 1B: Western blot showing replacement of endogenous WT with exogenous WT and X285K HOXB13 in representative clones (LN95WT clone 26 and LN95X285K clone 21). Higher molecular weights for the exogenous proteins are due to tags and 96 amino acids extension. Homogeneous RFP expression was confirmed upon Dox treatment.

FIG. 2A-2C. FIG. 2A: CCNB1 mRNA level was quantified by qRT-PCR in 44 samples from the 11 clones. All clones were treated with 20 ng/ml Dox and 1 nM R1881 as shown in FIG. 7A. Mean±SD values from four technical replicate are shown (see Methods for details). P-values for ANOVA were p<0.0001 for both R1881 (−) and 1 nM R1881. Only comparisons with p<0.05 from Turkey's multiple comparison tests are annotated in the graph. ****: p<0.0001, ***: p=0.0005, **: p<0.01. FIG. 2B: Heatmap of 67 differentially expressed genes between five LN95WT clones and three LN95X285K clones treated with Dox and R1881. CCNB1 and MYC are highlighted in bold. FIG. 2C: Heatmap of AR-induced genes in 44 samples from 11 clones. AR-induced genes were retrieved from reference 11 and divided into HOXB13-repressed genes (left) and HOXB13-activated genes (right).

FIG. 3A-3B. Western blot analyses comparing key proteins of the E2F/MYC signature in (FIG. 3A) WT and (FIG. 3B) X285K representative clones expressing similar levels of exogenous HOXB13 (LN95WT clone 26 and LN95X285K clone 21). Cells were treated with high and low dose ranges of Dox as indicated for two days with 1 nM R1881. B-actin was used as a loading control.

FIG. 4A-4D. FIG. 4A: Scatterplot with ChIP-Seq differential binding (log 2-fold change) in the x-axis and differential mRNA expression (log 2-fold change) in the y-axis. Upregulated genes in Dox+R1881+LN95X285K RNA-Seq with gained peaks in Dox+R1881+LN95X285K ChIP-Seq are presented as red dots. FIG. 4B: Integrated genome browser (IGV) snapshot of HOXB13 binding peaks at 5′ upstream region of CCNBI. X285K binding peaks are marked red. FIG. 4C, 4D: IGV snapshots showing HOXB13 binding peaks around PCAT1 and PCAT2 regions known as MYC super-enhancer. X285K binding peaks are marked red.

FIG. 5. Protein structures of WT and X285K HOXB13 by AlphaFold. MEIS interacting domains and homeodomain helices 1-3 are marked with green color, and the 96 amino acids (AA) elongations specific to X285K are marked in red. DNA and amino acid sequences are shown below around the stop codons (red asterisk) in the presence (WT) or absence (X285K) of c.853T (red box).

FIG. 6. Western blot and qRT-PCR results for HOXB 13 in five LN95WT, three X285K, and three NT clones were used in this study. Cells were treated with indicated concentrations of Dox for four days. Mean±SD values from two replicates are shown.

FIG. 7A-7C. FIG. 7A: Treatment schedule of 11 clones. Each clone generates four samples (with or without Dox, with or without R1881 as labeled). FIG. 7B: Volcano plot of RNA-Seq data comparing five LN95WT clones and three LN95X285K clones treated with Dox and R1881. CCNB1 and MYC are highlighted in bold. FIG. 7C: GSEA results comparing five LN95WT clones and three LN95X285K clones treated with Dox and R1881. NES: normalized enrichment score.

FIG. 8A-8C. FIG. 8A: Entire blot images of WT and X285K HOXB13 from FIG. 3A and B (LN95WT clone 26 and LN95X285K clone 21). To rule out dosage differences, the same amount of protein was loaded, and low-Dox-dose-range WT and X285K were developed next to each other with the same exposure time. FIG. 8B: High-Dox-dose-range WT and X285K were developed next to each other with the same exposure time. FIG. 8C: WT and X285K were run on the same blot.

FIG. 9A-9B. FIG. 9A: HOXB13 ChIP-Seq results. The number of peak regions (Y axis) stratified by peak annotations. FIG. 9B: HOXB13 ChIP-Seq analysis. Cells (LN95WT clone 26 and LN95X285K clone 21) were treated with 20 ng/ml Dox and 1 nM R1881 as shown in FIG. 7A. HOXB13 enrichment around peak regions in LN95WT and LN95X285K is shown. The upper panels show the average reads around peak regions. Purple: the average reads of gained peaks in WT, blue: the average reads of gained peaks in X285K. Lower panels show read density heatmaps classified by gained peaks in WT (top) or X285K (bottom).

FIG. 10. Clinical course of X285K carriers. X axis: months after diagnosis (dx). Y axis: Serum PSA values. Systemic treatment initiation timepoints are marked with blue dots. ADT: first-line androgen deprivation therapy, DTX: docetaxel, CBZ: cabazitaxel, ENZ: enzalutamide, ABI: abiraterone, CTC: circulating tumor cell.

FIG. 11. Location of variants in HOXB13.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Definitions

As used herein, the term “HOXB13 nucleic acid molecule” refers to a polynucleotide encoding a HOXB13 polypeptide. An exemplary HOXB13 nucleic acid molecule (mRNA) is provided at NCBI Accession No. NM_006361 (SEQ ID NO:13).

A “Homeobox B13 polypeptide” or “HOXB13 polypeptide” means a polypeptide or fragment thereof having at least 85% amino acid identity to NCBI Accession No. NP_006352 (SEQ ID NO:11) and having DNA binding activity. A HOXB13 polypeptide also means a polypeptide or fragment thereof having at least 85% amino acid identity to SEQ ID NO:13 (X285K variant)

The term “alteration” refers to any change in the nucleic acid or amino acid sequence of a molecule relative to a reference sequence. Such alteration may be, for example, a missense, frameshift or substitution mutation. The reference sequence is typically a wild-type HoxB13 nucleic acid or amino acid sequence.

“Clinical aggressiveness” refers to the severity of a neoplasia. Aggressive neoplasias are more likely to metastasize than less aggressive neoplasias. While conservative methods of treatment are appropriate for less aggressive neoplasias, more aggressive neoplasias require more aggressive therapeutic regimens.

The term “severity of neoplasia” means the degree of pathology. The severity of a neoplasia increases, for example, as the stage or grade of the neoplasia increases.

As used herein, “detect” refers to identifying the presence, absence, level, or concentration of an agent. In particular embodiments, “detect” refers to the presence of a HOXB13 alteration such as X285K (rs77179853).

“Detectable” means a moiety that when linked to a molecule of interest renders the latter detectable. Such detection may be via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

A “genotype” refers to the genetic composition of a cell, organism, or individual. With reference to the invention, the genotype of an individual is determined as heterozygous or homozygous for one or more variant alleles of interest. “Genotyping” refers to the characterization of the two alleles in one or more genes of interest (i.e., to determine a genotype).

The term “heterozygous” means that a chromosomal locus has two different alleles. In one embodiment of the invention, heterozygous refers to a genotype in which one allele has a wild-type HOXB13 sequence and the other allele has a sequence encoding, for example, the HOXB13 variant X285K (rs77179853).

“Homozygous” means that a chromosomal locus has two identical alleles. In the invention, homozygous wild-type is meant to refer to a genotype in which both alleles have a wild-type HOXB13 sequence. In some embodiments, homozygous can refer to a genotype in which both alleles have a sequence encoding the HOXB13 variant X285K (rs77179853).

As used herein, the term “propensity” means that a subject has an increased risk of developing disease relative to a reference subject. Such an increased risk is associated with the presence of an alteration in a HoxB13 nucleic acid or amino acid sequence that predisposes the subject to develop prostate cancer relative to the risk of prostate cancer in a reference subject carrying a wild-type HoxB13 sequence. In particular embodiments, “propensity” refers to an increased risk of developing aggressive prostate cancer relative to a reference subject, which can include non-aggressive cancer or no cancer.

A “reference” refers a standard of comparison. For example, the nucleotide sequence in a patient sample may be compared to the nucleotide sequence present in a corresponding healthy cell or tissue.

A “positive family history” refers to the presence of prostate cancer is a first degree relative (e.g., son, father, uncle, brother).

“Periodic” means at regular intervals. Periodic patient monitoring includes, for example, a schedule of tests that are administered daily, bi-weekly, bi-monthly, monthly, bi-annually, or annually.

A “marker” refers to any protein or polynucleotide having an alteration in activity, expression level, or sequence that is associated with a disease, disorder, or condition. In certain embodiments, a “marker” refers to a patient DNA sample having the HOXB13 variant X285K (rs77179853).

A “marker profile” refers to a characterization of the expression or expression level of two or more polypeptides or polynucleotides.

As used herein a “nucleic acid or oligonucleotide probe” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences lacking complete complementarity with the probe sequence depending upon the stringency of the hybridization conditions. The probes can be directly labeled with isotopes, for example, chromophores, lumiphores, chromogens, or indirectly labeled with biotin to which a streptavidin complex may later bind. By assaying for the presence or absence of the probe, one can detect the presence or absence of a target gene of interest.

The term “specifically binds” refers to a compound or antibody that recognizes and binds a polypeptide of the invention, but which does not substantially recognize and bind other molecules in a sample.

The phrase “selectively (or specifically) hybridizes to” refers to the binding, duplexing, or hybridizing of a molecule only to a particular nucleotide sequence under stringent hybridization conditions when that sequence is present in a complex mixture (for example, total cellular or library DNA or RNA).

As used herein, a “single nucleotide polymorphism” or “SNP” refers to a DNA sequence variation occurring when a single nucleotide in the genome differs between members of a biological species or paired chromosomes in an individual. SNPs are used as genetic markers for variant alleles. In particular embodiments, a SNP refers to the HOXB13 variant X285K (rs77179853).

A “target nucleic acid molecule” means a nucleic acid or biomarker of the sample that is to be detected.

As used herein, a “variant” refers to a polynucleotide or polypeptide sequence that differs from a wild-type or reference sequence by one or more nucleotides or one or more amino acids. An exemplary HOXB13 variant includes X285K (rs77179853).

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency.

As used herein, “substantially identical” refers to a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, a “subject” means a human or animal. The terms, “patient”, “individual” and “subject” are used interchangeably herein. In specific embodiments, the subject is mammal. In various embodiments, the subject, patient or individual is human.

A subject can be one who has been previously diagnosed with or identified as suffering from or having a condition, disease, or disorder in need of treatment (e.g., prostate cancer) or one or more complications related to the condition, disease, or disorder, and optionally, have already undergone treatment for the condition, disease, disorder, or the one or more complications related to the condition, disease, or disorder. Alternatively, a subject can also be one who has not been previously diagnosed as having prostate cancer or one or more complications related to prostate cancer. For example, a subject can be one who exhibits one or more risk factors for prostate cancer, or one or more complications related thereto, or a subject who does not exhibit risk factors. A “subject in need” of treatment for prostate cancer can be a subject suspected of having prostate cancer, diagnosed as having prostate cancer, already treated or being treated for prostate cancer, not treated for prostate cancer, or at risk of developing prostate cancer.

In some embodiments, the subject is selected from the group consisting of a subject suspected of having prostate cancer, a subject that has prostate cancer, a subject diagnosed with prostate cancer, a subject that has non-aggressive prostate cancer, a subject suspected of having aggressive prostate cancer, a subject that has been treated for prostate cancer, a subject that is being treated for prostate cancer, and a subject that is at risk of developing prostate cancer.

The term “one or more of” refers to combinations of various biomarkers. The term encompasses 1, 2, 3, 4, 5, 6, 7, 8, 9, 10, 11, 12, 13, 15, 16, 17, 18, 19, 20, 21, 22, 23, 24, 25, 26, 27, 28, 29, 30, 31, 32, 33, 34, 35, 36, 37, 38, 39, 40 . . . N, where “N” is the total number of biomarker proteins in the particular embodiment. The term also encompasses, and is interchangeably used with, at least 1, at least 2, at least 3, at least 4, at least 5, at least 6, at least 7, at least 8, at least 9, at least 10, at least 11, at least 12, at least 13, at least 15, 16, 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40 . . . N. It is understood that the recitation of biomarkers herein includes the phrase “one or more of” the biomarkers and, in particular, includes the “at least 1, at least 2, at least 3” and so forth language in each recited embodiment of a biomarker panel.

As used herein, the terms “treat”, “treatment”, “treating”, or “amelioration” when used in reference to a disease, disorder or medical condition (e.g., prostate cancer), refer to both therapeutic treatment and prophylactic or preventative measures, wherein the object is to reverse, alleviate, ameliorate, inhibit, lessen, slow down or stop the progression or severity of a symptom, a condition, a disease, or a disorder. The term “treating” includes reducing or alleviating at least one adverse effect or symptom of prostate cancer. Treatment is generally “effective” if one or more symptoms or clinical markers are reduced. Alternatively, treatment is “effective” if the progression of prostate cancer is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing prostate cancer even if the treatment is ultimately unsuccessful. Those in need of treatment include those already with prostate cancer as well as those prone to have prostate cancer or those in whom prostate cancer is to be prevented.

Non-limiting examples of treatments or therapeutic treatments include pharmacological or biological therapies and/or interventional surgical treatments.

As used herein, the term “administering,” refers to the placement an agent or a treatment as disclosed herein into a subject by a method or route which results in at least partial localization of the agent or treatment at a desired site. “Route of administration” may refer to any administration pathway known in the art, including but not limited to aerosol, nasal, via inhalation, oral, anal, intra-anal, peri-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrastemai, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions can be in the form of aerosol, lotion, cream, gel, ointment, suspensions, solutions or emulsions. In accordance with the present invention, “administering” can be self-administering. For example, it is considered as “administering” that a subject consumes a composition as disclosed herein.

The term “% identical” between two polypeptide or polynucleotide sequences refers to the number of identical matched positions shared by the sequences over a comparison window, considering additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. The percentage of sequence identity is calculated by determining the number of positions at which the identical amino acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is b12seq, part of the BLAST suite of program available from the U.S. government's National Center for Biotechnology Information BLAST web site. B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa. In certain embodiments, the percentage identity “X” of a first amino acid sequence to a second sequence amino acid is calculated as 100x (Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence. One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2 (ClustalX is a version of the ClustalW2 program ported to the Windows environment). Another suitable program is MUSCLE. ClustalW2 and MUSCLE are alternatively available, e.g., from the European Bioinformatics Institute (EBI).

The term “therapeutic agent” refers to any biological or chemical agent used in the treatment of a disease or disorder. Therapeutic agents include any suitable biologically active chemical compounds, biologically derived components such as cells, peptides, antibodies, and polynucleotides, and radiochemical therapeutic agents such as radioisotopes. In some embodiments, the therapeutic agent comprises a chemotherapeutic agent or an analgesic.

### II. Detection/Measurement of Nucleic Acid Markers

The present invention provides a number of diagnostic assays that are useful for the identification or characterization of prostate cancer in a subject. Such methods may be used alone or in combination with standard methods for monitoring a subject for prostate cancer. In one embodiment, a subject is identified as being at risk of developing prostate cancer by the presence of the SNP rs77179853 (corresponding to the genetic variant HOXB13 X285K), alone or in combination with other standard methods. To determine the stage or grade of a neoplasia, grading is used to describe how abnormal or aggressive the neoplastic cells appear, while staging is used to describe the extent of the neoplasia. In certain embodiments, the grade and stage of the neoplasia in combination with the presence of the SNP rs77179853 (corresponding to the genetic variant HOXB13 X285K) is used to determine a subject's long-term prognosis (i.e., probable response to treatment and survival). Thus, the methods of the invention are useful for predicting a patient's prognosis, and for selecting a course of treatment.

### A. Types of Biological Samples

The presence of SNP rs77179853 (corresponding to the genetic variant HOXB13 X285K) can be detected in different types of biologic samples. In one embodiment, the biologic sample is a tissue sample that includes cells of a tissue or organ (e.g., prostatic tissue cells). Prostatic tissue is obtained, for example, from a biopsy of the prostate. In another embodiment, the biologic sample is a biologic fluid sample. Biological fluid samples include blood, blood serum, plasma, urine, seminal fluids, and ejaculate, or any other biological fluid useful in the methods of the invention.

### B. Genotyping of HOXB13 Polymorphisms

In particular embodiments, a HOXB13 isoform is amplified by PCR to determine the genotype of the isoform, e.g., HOXB13 X285K. The amplified nucleic acid corresponding to HOXB13 may be analyzed using a variety of methods for detecting variant alleles to determine the genotype. The presence or absence of a polymorphism (e.g., X285K) in the HOXB13 gene may be evaluated using various techniques. For example, the HOXB13 gene is amplified by PCR and sequenced to determine the presence or absence of a single nucleotide polymorphism (SNP). In certain embodiments, real-time PCR may be used to detect a single nucleotide polymorphism of the amplified products. In other embodiments, a polymorphism in the amplified products may be detected using a technique including hybridization with a probe specific for a single nucleotide polymorphism, restriction endonuclease digestion, primer extension, microarray or gene chip analysis, mass spectrometry, or a DNAse protection assay.

Various PCR testing platforms that may be used with the present invention include: 5′ nuclease (TaqMan® probes), molecular beacons, and FRET hybridization probes. These detection methods rely on the transfer of light energy between two adjacent dye molecules, a process referred to as fluorescence resonance energy transfer.

In certain embodiments, a 5′ nuclease probe may be used to detect a polymorphism of the present invention. 5′ nuclease probes are often referred to by the proprietary name, TaqMan® probes. A TaqMan® probe is a short oligonucleotide (DNA) that contains a 5′ fluorescent dye and 3′ quenching dye. To generate a light signal (i.e., remove the effects of the quenching dye on the fluorescent dye), two events must occur. First, the probe must bind to a complementary strand of DNA, e.g., at about 60° C. Second, at this temperature, Taq polymerase, which is commonly used for PCR, must cleave the 5′ end of the TaqMan® probe (5′ nuclease activity), separating the fluorescent dye from the quenching dye.

In order to differentiate a single nucleotide polymorphism from a wild-type sequence in the DNA from a subject, a second probe with complementary nucleotide(s) to the polymorphism and a fluorescent dye with a different emission spectrum are typically utilized. Thus, these probes can be used to detect a specific, predefined polymorphism under the probe in the PCR amplification product. Two reaction vessels are typically used, one with a complementary probe to detect wild-type target DNA and another for detection of a specific nucleic acid sequence of a mutant strain. Because TaqMan® probes typically require temperatures of about 60° C. for efficient 5′ nuclease activity, the PCR may be cycled between about 90-95° C. and about 60° C. for amplification. In addition, the cleaved (free) fluorescent dye can accumulate after each PCR temperature cycle; thus, the dye can be measured at any time during the PCR cycling, including the hybridization step. In contrast, molecular beacons and FRET hybridization probes typically involve the measurement of fluorescence during the hybridization step.

Genotyping for the X285K polymorphism in the HOXB13 gene may be evaluated using the following (5′ endonuclease probe) real-time PCR technique. Genotyping assays can be performed in duplicate and analyzed on a Bio-Rad iCycler Iq® Multicolor Real-time detection system (Bio-Rad Laboratories, Hercules, Calif.). Real-time polymerase chain reaction (PCR) allelic discrimination assays to detect the presence or absence of specific single nucleotide polymorphisms in a HOXB13 gene, Gly 143Glu (genomic: nt 9486; Cdna: nt 428) and Asp260fs (genomic: nt 12754; Cdna: nt 780), may utilize fluorogenic TaqMan® Probes.

Real-time PCR amplifications may be carried out in a 10 μl reaction mix containing 5 ng genomic DNA, 900 Nm of each primer, 200 Nm of each probe and 5 μl of 2.times. TaqMan® Universal PCR Master Mix (contains PCR buffer, passive reference dye ROX, deoxynucleotides, uridine, uracil-N-glycosylase and AmpliTaq Gold DNA polymerase; Perkin-Elmer, Applied Biosystems, Foster City, Calif.). Cycle parameters may be: 95° C. for 10 min, followed by 50 cycles of 92° C. for 15 sec and 60° C. for 1 min. Real-time fluorescence detection can be performed during the 60° C. annealing/extension step of each cycle. The IQ software may be used to plot and automatically call genotypes based on a two parameter plot using fluorescence intensities of FAM and VIC at 49 cycles.

### C. Molecular Beacons

Molecular beacons are another real-time PCR approach which may be used to identify the presence or absence of a polymorphism of the present invention. Molecular beacons are oligonucleotide probes that are labeled with a fluorescent dye (typically on the 5′ end) and a quencher dye (typically on the 3′ end). A region at each end of the molecular beacon probe is designed to be complementary to itself, so at low temperatures the ends anneal, creating a hairpin structure. This hairpin structure positions the two dyes in close proximity, quenching the fluorescence from the reporter dye. The central region of the probe is designed to be complementary to a region of a PCR amplification product. At higher temperatures, both the PCR amplification product and probe are single stranded. As the temperature of the PCR is lowered, the central region of the molecular beacon probe may bind to the PCR product and force the separation of the fluorescent reporter dye from the quenching dye. Without the quencher dye in close proximity, a light signal from the reporter dye can be detected. If no PCR amplification product is available for binding, the probe can re-anneal to itself, bringing the reporter dye and quencher dye into close proximity, thus preventing fluorescent signal.

Two or more molecular beacon probes with different reporter dyes may be used for detecting single nucleotide polymorphisms. For example, a first molecular beacon designed with a first reporter dye may be used to indicate the presence of a SNP and a second molecular beacon designed with a second reporter dye may be used to indicate the presence of the corresponding wild-type sequence; in this way, different signals from the first and/or second reporter dyes may be used to determine if a subject is heterozygous for a SNP, homozygous for a SNP, or homozygous wild-type at the corresponding DNA region. By selection of appropriate PCR temperatures and/or extension of the probe length, a molecular beacons may bind to a target PCR product when a nucleotide polymorphism is present but at a slight cost of reduced specificity. Molecular beacons advantageously do not require thermocycling, so temperature optimization of the PCR is simplified.

### D. FRET Hybridization Probes

FRET hybridization probes, also referred to as LightCycler® probes, may also be used to detect a polymorphism of the present invention. FRET hybridization probes typically comprise two DNA probes designed to anneal next to each other in a head-to-tail configuration on the PCR product. Typically, the upstream probe has a fluorescent dye on the 3′ end and the downstream probe has an acceptor dye on the 5′ end. If both probes anneal to the target PCR product, fluorescence from the 3′ dye can be absorbed by the adjacent acceptor dye on the 5′ end of the second probe. As a result, the second dye is excited and can emit light at a third wavelength, which may be detected. If the two dyes do not come into close proximity in the absence of sufficient complimentary DNA, then FRET does not occur between the two dyes. The 3′ end of the second (downstream) probe may be phosphorylated to prevent it from being used as a primer by Taq during PCR amplification. The two probes may encompass a region of 40 to 50 DNA base pairs.

FRET hybridization probe technology permits melting curve analysis of the amplification product. If the temperature is slowly raised, probes annealing to the target PCR product will be reduced and the FRET signal will be lost. The temperature at which half the FRET signal is lost is referred to as the melting temperature of the probe system. A single nucleotide polymorphism in the target DNA under a hybridization FRET probe will still generate a signal, but the melting curve will display a lower Tm. The lowered Tm can indicate the presence of a specific polymorphism. The target PCR product is detected and the altered Tm informs the user there is a difference in the sequence being detected. Like molecular beacons, there is not a specific thermocycling temperature requirement for FRET hybridization probes. Like molecular beacons, FRET hybridization probes have the advantage of being recycled or conserved during PCR temperature cycling, and a fluorescent signal does not accumulate as PCR product accumulates after each PCR cycle.

### E. Primer Extension

Primer extension is another technique which may be used according to the present invention. A primer and no more than three NTPs may be combined with a polymerase and the target sequence, which serves as a template for amplification. By using less than all four NTPs, it is possible to omit one or more of the polymorphic nucleotides needed for incorporation at the polymorphic site. It is important for the practice of the present invention that the amplification be designed such that the omitted nucleotide(s) is (are) not required between the 3′ end of the primer and the target polymorphism. The primer is then extended by a nucleic acid polymerase, in a preferred embodiment by Taq polymerase. If the omitted NTP is required at the polymorphic site, the primer is extended up to the polymorphic site, at which point the polymerization ceases. However, if the omitted NTP is not required at the polymorphic site, the primer will be extended beyond the polymorphic site, creating a longer product. Detection of the extension products is based on, for example, separation by size/length which will thereby reveal which polymorphism is present.

### F. RFLP

Restriction Fragment Length Polymorphism (RFLP) is a technique in which different DNA sequences may be differentiated by analysis of patterns derived from cleavage of that DNA. If two sequences differ in the distance between sites of cleavage of a particular restriction endonuclease, the length of the fragments produced will differ when the DNA is digested with a restriction enzyme. The similarity of the patterns generated can be used to differentiate species (and even strains) from one another.

Restriction endonucleases in turn are the enzymes that cleave DNA molecules at specific nucleotide sequences depending on the particular enzyme used. Enzyme recognition sites are usually 4 to 6 base pairs in length. Generally, the shorter the recognition sequence, the greater the number of fragments generated. If molecules differ in nucleotide sequence, fragments of different sizes may be generated. The fragments can be separated by gel electrophoresis. Restriction enzymes are isolated from a wide variety of bacterial genera and are thought to be part of the cell's defenses against invading bacterial viruses. Use of RFLP and restriction endonucleases in SNP analysis requires that the SNP affect cleavage of at least one restriction enzyme site.

### G. Mass Spectrometry

Mass spectrometry may also be used to detect a polymorphism of the present invention. By exploiting the intrinsic properties of mass and charge, mass spectrometry (MS) can resolve and confidently identify a wide variety of complex compounds. Traditional quantitative MS has used electrospray ionization (ESI) followed by tandem MS (MS/MS) while other methods utilize matrix assisted laser desorption/ionization (MALDI) followed by time of flight (TOF) MS. Methods of mass spectroscopy that may be used with the present invention include: ESI, ESI tandem mass spectroscopy (ESI/MS/MS), Secondary ion mass spectroscopy (SIMS), Laser desorption mass spectroscopy (LD-MS), Laser Desorption Laser Photoionization Mass Spectroscopy (LDLPMS), and MALDI-TOF-MS.

### H. Microarrays

The invention provides diagnostic microarrays for detecting the SNP rs77179853 (corresponding to the genetic variant HOXB13 X285K) in a biological sample. HOXB13 nucleic acid molecules or polypeptides are useful as hybridizable array elements in the microarray. The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid and polypeptide microarrays are known to the skilled.

### I. Antibodies

Antibodies that selectively bind a variant HOXB13 polypeptide (e.g., X285K) are useful in the methods of the invention. As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′) 2, and Fab. F(ab′) 2, and Fab fragments that lack the Fc fragment of intact antibody, clear more rapidly from the circulation, and may have less non-specific tissue binding of an intact antibody. The antibodies of the invention also comprise whole native antibodies, bispecific antibodies; chimeric antibodies; Fab, Fab′, single chain V region fragments (scFv), fusion polypeptides, nanobodies, linear antibodies and antibodies having multiple valencies (e.g., diabodies, tribodies, tetrabodies and pentabodies).

### J. Sequencing

Nucleic acids may be sequenced using sequencing methods such as next-generation sequencing, high-throughput sequencing, massively parallel sequencing, sequencing-by-synthesis, paired-end sequencing, single-molecule sequencing, nanopore sequencing, pyrosequencing, semiconductor sequencing, sequencing-by-ligation, sequencing-by-hybridization, RNA-Seq, Digital Gene Expression, Single Molecule Sequencing by Synthesis (SMSS), Clonal Single Molecule Array (Solexa), shotgun sequencing, Maxim-Gilbert sequencing, primer walking, and Sanger sequencing.

Sequencing methods may comprise targeted sequencing, whole-genome sequencing (WGS), lowpass sequencing, bisulfite sequencing, whole-genome bisulfite sequencing (WGBS), or a combination thereof. Sequencing methods may include preparation of suitable libraries. Sequencing methods may include amplification of nucleic acids (e.g., by targeted or universal amplification, such as PCR).

Sequencing reads can be obtained from various sources including, for example, whole genome sequencing, whole exome-sequencing, targeted sequencing, next-generation sequencing, pyrosequencing, sequencing-by-synthesis, ion semiconductor sequencing, tag-based next generation sequencing semiconductor sequencing, single-molecule sequencing, nanopore sequencing, sequencing-by-ligation, sequencing-by-hybridization, Digital Gene Expression (DGE), massively parallel sequencing, Clonal Single Molecule Array (Solexa/Illumina), sequencing using PacBio, and Sequencing by Oligonucleotide Ligation and Detection (SOLID).

In some embodiments, sequencing comprises modification of a nucleic acid molecule or fragment thereof, for example, by ligating a barcode, a unique molecular identifier (UMI), or another tag to the nucleic acid molecule or fragment thereof. Ligating a barcode, UMI, or tag to one end of a nucleic acid molecule or fragment thereof may facilitate analysis of the nucleic acid molecule or fragment thereof following sequencing. In some embodiments, a barcode is a unique barcode (i.e., a UMI). In specific embodiments, a barcode is non-unique, and barcode sequences can be used in connection with endogenous sequence information such as the start and stop sequences of a target nucleic acid (e.g., the target nucleic acid is flanked by the barcode and the barcode sequences, in connection with the sequences at the beginning and end of the target nucleic acid, creates a uniquely tagged molecule).

Sequencing reads may be processed using methods such as de-multiplexing, de-deduplication (e.g., using unique molecular identifiers, UMIs), adapter-trimming, quality filtering, GC correction, amplification bias correction, correction of batch effects, depth normalization, removal of sex chromosomes, and removal of poor-quality genomic bins.)

In various embodiments, sequencing reads may be aligned to a reference nucleic acid sequence. In one example, the reference nucleic acid sequence is a human reference genome. As examples, the human reference genome can be hg19, hg38, GrCH38, GrCH37, NA12878, or GM12878.

### III. Treatment Methods

In another aspect, the present invention provides a prostate cancer therapy or therapeutic interventions practically applied following the measurement/detection of biomarkers. In particular embodiments, therapeutic intervention comprises prostatectomy, radiation therapy, cryotherapy (also referred to as cryosurgery or cryoablation), hormone therapy, chemotherapy, immunotherapy and combinations thereof.

Prostatectomy includes radical prostatectomy (open (radical retropubic prostatectomy or radical perineal prostatectomy) or lateral (laparoscopic radical prostatectomy including robotic-assisted), and transurethral resection of the prostate (TURP).

Radiation therapy includes external beam radiation (three-dimensional conformal radiation therapy (3D-CRT), intensity modulated radiation therapy (IMRT), stereotactic body radiation therapy (SBRT), proton beam radiation therapy) and brachytherapy (internal radiation) (permanent (low dose rate or LDR) brachytherapy or temporary (high dose rate or HDR) brachytherapy).

Hormone therapy (androgen suppression therapy) includes orchiectomy (surgical castration), luteinizing hormone-release hormone (LHRH) agonists (e.g., leuprolide, goserelin, triptorelin, histrelin), LHRH antagonists (e.g., degarelix), treatment to lower androgen levels from the adrenal glands (e.g., abiraterone, ketoconazole), anti-androgens (e.g., flutamide, bicalutamide, nilutamide, enzalutamide, apalutamide), and estrogens.

Chemotherapy includes treatment with compounds including, but not limited to, docetaxel, cabazitaxel, mitoxantrone, and estramustine.

Immunotherapy includes, but is not limited to, a cancer vaccine (e.g., sipuleucel-T), as well as immune checkpoint inhibitors (e.g., PD-1 inhibitors including pembrolizumab). Illustrative immune checkpoint inhibitors include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor).

A prostate therapeutic intervention can comprise a targeted therapy including poly (ADP)-ribose polymerase (PARP) inhibitor (e.g., niraparib (zejula), olaparib (lynparza), and rucaparib (rubraca)).

Other therapeutic interventions for prostate cancer include an androgen receptor (AR)-targeted therapy (e.g., enzalutamide, ARN-509, ODM-201, EPI-001, hydrazinobenzoylcurcumin (HBC), aberaterone, geleterone, and seviteronel), an antimicrotubule agent, an alkylating agent and an anthracenedione.

In particular embodiments, a therapeutic intervention for prostate cancer can include the administration of drugs including, but not limited to, Abiraterone Acetate, Apalutamide, Bicalutamide, Cabazitaxel, Casodex (Bicalutamide), Darolutamide, Degarelix, Docetaxel, Eligard (Leuprolide Acetate), Enzalutamide, Erleada (Apalutamide), Firmagon (Degarelix), Flutamide, Goserelin Acetate, Jevtana (Cabazitaxel), Leuprolide Acetate, Lupron (Leuprolide Acetate), Lupron Depot (Leuprolide Acetate), Lynparza (Olaparib), Mitoxantrone Hydrochloride, Nilandron (Nilutamide), Nilutamide, Nubeqa (Darolutamide), Olaparib, Provenge (Sipuleucel-T), Radium 223 Dichloride, Rubraca (Rucaparib Camsylate), Rucaparib Camsylate, Sipuleucel-T, Taxotere (Docetaxel), Xofigo (Radium 223 Dichloride), Xtandi (Enzalutamide), Zoladex (Goserelin Acetate), Zytiga (Abiraterone Acetate).

### IV. Detection Kits

In another aspect, the present invention provides kits for detecting one or more biomarkers including the HOXB13 alteration X285K (rs77179853). The exact nature of the components configured in the inventive kit depends on its intended purpose. In one embodiment, the kit is configured particularly for human subjects.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

In various embodiments, the present invention provides a kit comprising: (a) one or more internal standards suitable for the detection of one or more biomarkers including by any one or more of mass spectrometry, antibody method, antibodies, lectins, nucleic acid aptamer method, nucleic acid aptamers, immunoassay, ELISA, immunoprecipitation, SISCAPA, Western blot, PCR (qPCR, digital PCR, etc.) or combinations thereof; and (b) reagents and instructions for sample processing, preparation and biomarker measurement/detection. The kit can further comprise (c) instructions for using the kit to detect biomarkers in a sample obtained from the subject.

A nucleic acid-based detection kit may include a primer or probe that specifically hybridizes to a target polynucleotide (e.g., HOXB13 alteration such as X285K (rs77179853)). The kit can further include a target biomarker polynucleotide to be used as a positive control. Also included may be enzymes suitable for amplifying nucleic acids including various polymerases (reverse transcriptase, Taq, Sequenase™, DNA ligase etc., depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe.

In a more specific embodiment, the kit is provided as a PCR kit comprising primers that specifically bind to one or more of the nucleic acid biomarkers described herein. The kit can further comprise substrates and other reagents necessary for conducting PCR (e.g., quantitative real-time PCR, digital PCR). The kit can be configured to conduct singleplex or multiplex PCR. The kit can further comprise instructions for carrying out the PCR reaction(s). In specific embodiments, the biological sample obtained from a subject may be manipulated to extract nucleic acid. In a further embodiment, the nucleic acids are contacted with primers that specifically bind the target biomarkers to form a primer: biomarker complex. The complexes can then be amplified and detected.

The reagents described herein, which may be optionally associated with detectable labels, can be presented in the format of a microfluidics card, a chip or chamber, a microarray or a kit adapted for use with the assays described in the examples or below, e.g., RT-PCR, Q PCR, digital PCR techniques described herein.

In other embodiments, the kit comprises reagents necessary for processing of samples and performance of an immunoassay such as an ELISA. Thus, in certain embodiments, the kit comprises a substrate for performing the assay (e.g., a 96-well polystyrene plate). The substrate can be coated with antibodies specific for a biomarker protein (HOXB13 alteration such as X285K (rs77179853)). In a further embodiment, the kit can comprise a detection antibody including, for example, a polyclonal antibody specific for a biomarker protein conjugated to a detectable moiety or label (e.g., horseradish peroxidase). The kit can also comprise a standard, e.g., a human protein standard. The kit can also comprise one or more of a buffer diluent, calibrator diluent, wash buffer concentrate, color reagent, stop solution and plate sealers (e.g., adhesive strip).

In particular embodiments, the kit may comprise a solid support, such as a chip, microtiter plate (e.g., a 96-well plate), bead, or resin having protein biomarker capture reagents attached thereon. The kit may further comprise a means for detecting the protein biomarkers, such as antibodies, and a secondary antibody-signal complex such as horseradish peroxidase (HRP)-conjugated goat anti-rabbit IgG antibody and tetramethyl benzidine (TMB) as a substrate for HRP. In other embodiments, the kit can comprise magnetic beads conjugated to the antibodies (or separate containers thereof for later conjugation). The kit can further comprise detection antibodies, for example, biotinylated antibodies or lectins that can be detected using, for example, streptavidin labeled fluorescent markers such as phycoerythrin. The kit can be configured to perform the assay in a singleplex or multiplex format.

The kit may be provided as an immuno-chromatography strip comprising a membrane on which the antibodies are immobilized, and a means for detecting, e.g., gold particle bound antibodies, where the membrane, includes NC membrane and PVDF membrane. The kit may comprise a plastic plate on which a sample application pad, gold particle bound antibodies temporally immobilized on a glass fiber filter, a nitrocellulose membrane on which antibody bands and a secondary antibody band are immobilized and an absorbent pad are positioned in a serial manner, so as to keep continuous capillary flow of the sample.

In a specific embodiment, a kit comprises one or more of (a) magnetic beads for conjugating to antibodies that specifically bind biomarker proteins of interest; (b) monoclonal antibodies that specifically bind the biomarker proteins of interest; (c) biotinylated immunoglobulin G detection antibodies; (d) biotinylated lectins that specifically bind the biomarker proteins of interest; and (e) streptavidin labeled fluorescent marker.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: The HOXB13 Variant X285K is Associated with Clinical Significance And Early Age at Diagnosis in African American Prostate Cancer Patients

BACKGROUND: Recently, a novel HOXB13 variant (X285K) was observed in men of African descent with prostate cancer (PCa) in Martinique. Little is known about this or other variants in HOXB13 which may play a role in PCa susceptibility in African-American (AA) men.

METHODS: The present inventors sequenced HOXB 13 in an AA population of 1048 men undergoing surgical treatment for PCa at Johns Hopkins Hospital.

RESULTS: Seven non-synonymous germline variants were observed in the patient population. While six of these variants were seen only once, X285K was found in eight patients. In a case-case analysis, the present inventors find that carriers of this latter variant are at increased risk of clinically significant PCa (1.2% carrier rate in Gleason Score ≥7 PCa vs. 0% in Gleason Score <7 PCa, odds ratio, OR=inf; 95% Confidence Interval, 95% CI: 1.05-inf, P=0.028), as well as PCa with early age at diagnosis (2.4% carrier rate in patients <50 year vs. 0.5% carrier rate in patients ≥50 year, OR=5.25, 95% CI: 1.00-28.52, P=0.03).

CONCLUSIONS: While this variant is rare in the AA population (~0.2% MAF), its ancestry-specific occurrence and apparent preferential association with risk for the more aggressive disease at an early age emphasizes its translational potential as an important, novel PCa susceptibility marker in the high-risk AA population.

### Introduction

The present inventors hypothesized that there may exist currently uncharacterized, recurrent, function-altering variants in the HOXB13 gene that may play an important role in PCa susceptibility, particularly for early-onset disease, in African Americans. In this study, the present inventors examine a number of AA PCa patients for the occurrence of X285K and other variants in HOXB13 which may confer increased risk in this high-risk population.

### Results

To search for germline coding sequence variants in HOXB13 in a large, well-characterized African-American PCa population, the present inventors sequenced both exons of the gene in 1048 AA men who had undergone radical prostatectomy (RP) for treatment of PCa at the Brady Urological Institute between 2006 and 2018. Demographic characteristics and baseline clinical information are summarized in Table 1. The average age at diagnosis was 56.8 years old. The total number of cases with pathologic GS≥7 was 650, while 397 cases had GS≤6. For the pathologic stage, 700 cases (66.8%) were T2N0MX, with 284 cases (28.1%) T3aN0MX or T3b N0MX; 32 cases (3.1%) were N1.

The present inventors identified seven different rare non-synonymous changes in HOXB13: G84E, S93A, C100Y, L106R, P134Q, T2421 and X285K. All variants except X285K were observed only once in the study population. With the exception of S93A, all missense variants are predicted to be damaging or possibly damaging by SIFT and/or Polyphen2. T242I affects a conserved amino acid at the beginning of the second alpha helix in the DNA-binding homeodomain. P134Q is adjacent to a conserved domain harboring a putative binding site for MEIS homeobox cofactors. Table 2 lists a summary of these variants, and FIG. 11 shows the position of the variants.

X285K was the only recurrent non-synonymous change observed, seen in 8 AA cases (carrier frequency 0.76%). An examination of the pathologic variables in these cases revealed that all carriers of X285K had cancers with Gleason Score (GS) 7 (3+4, n=4 and 4+3, n=3), or GS 9 (4+5, n=1), Table 3. In all cases with GS>7 (n-650), the carrier frequency of X285K was 1.2% (n=8), whereas in the 395 cases with GS≤6, no carriers were observed; (odds ratio, OR=inf; 95% confidence interval, 95% CI: 1.05-inf, P=0.028, Table 4. In addition to the higher grade, X285K was significantly associated with earlier age at diagnosis of PCa. The median age of diagnosis in X285K carriers was 50.0 years (interquartile range, IQR: 42.0-63.0 years) vs. 57.0 years (IQR: 52.0-62.0 year) in non-carriers. The carrier rate of this variant in PCa cases with age at diagnosis <50 years was 2.4% (4 out of 170), which was significantly higher than the carrier rate (0.5%, 4 out of 878) in PCa with age at diagnosis ≥50 years (OR=5.25, 95% CI: 1.00-28.52, P-0.03, Table 5).

Frequency data available for X285K (rs77179853) from population databases demonstrated a consistently low minor allele fraction (MAF) in AA populations (Table 2, MAF 0.22% in gnomAD v.3 genomes, n=42,030 alleles). Compared to this AA population data, the frequency of X285K was non-significantly higher in AA PCa cases overall (MAF 0.38%, OR=1.74, 95% CI: 0.73-3.59, P=0.15), but significantly higher in cases with GS≥7 (MAF 0.63%, OR=2.86, 95% CI: 1.20-5.90, P=0.01).

### Discussion

To the best of our knowledge, this study is the largest HOXB 13 sequencing analysis in AA PCa patients published to date. The present inventors found that the X285K variant was: (1) the only recurrent HOXB13 variant in the AA study cohort, (2) associated with earlier age at diagnosis of PCa; and (3) associated with GS≥7 PCa in AA.

HOXB13 codes for a highly prostate-specific transcription factor that is necessary for normal prostate development [1. Its expression is maintained throughout adulthood, and during the initiation and progression of most prostate cancers. HOXB13 interacts with AR to modulate the expression of various androgen responsive genes, and this interaction with normal and variants of AR (e.g., ARv7) has been proposed to play a key role in reprograming the cistrome in both primary and metastatic PCa [14-1. However, a comprehensive understanding of the role that HOXB13 plays in prostate biology has not been described.

Despite extensive studies characterizing HOXB13 germline variants in PCa in men of European and Asian descent, little is known about possible associations of such variants in men of African descent. In this study, the present inventors sequenced the coding region of HOXB 13 in germline DNA from a well-characterized population of 1048 AA men undergoing surgical treatment for clinically localized PCa. This analysis revealed a set of six non-recurrent missense variants, and one recurrent, non-synonymous change. Of the missense changes, five were predicted to be deleterious. Five changes are in exon 1, coding for the amino-terminal portion of HOXB13, with the remaining change in the DNA-binding homeobox domain, coded for in exon 2. Whether these non-recurrent missense changes contribute to prostate carcinogenesis is unknown. Much larger studies will be required to determine their possible statistical association with the risk of PCa.

One of the missense variants observed is the Nordic founder variant, G84E. Its presence in PCa cases of African descent has been previously reported by others including Witte et al. who demonstrated that the variant was on a haplotype of European origin, indicating that it was most likely the result of population admixture rather than an independent variant event [1.

The only recurrent change observed was a frameshift variant resulting in loss of the in-frame stop codon due to a deletion of one nucleotide, c.853delT at the cDNA level and resulting in p. Ter285KextX95 (aka X285K) at the protein level. The functional significance of this stop loss is unclear at present, although there are multiple examples where stop-loss variants confer functional consequences, typically via instability of either the mRNA transcript or resultant protein [1. For the change described here, if translated, the stop-loss-containing mRNA would code for a HOXB 13 protein that extended an additional 96 amino acids before reaching the next in-frame stop codon. Whether this C-terminal protein extension, immediately 3′ to the DNA-binding homeodomain, affects HOXB13 protein function, or leads to instability of the transcript and/or protein remains to be determined. Clinvar lists this variant as having uncertain significance (two entries), or likely benign (one entry), although no stability or functional studies of this variant have been reported.

The c.853delT variant was first reported by Akbari et al. in a study of 1843 PCa cases and 2225 controls [. One of 200 AA cases and one of 160 AA controls carried the variant. It was absent from EA and Asian cases and controls. In our experience sequencing HOXB13 in germline DNA from over 5000 PCa cases of European descent, the present inventors have found only one example of X285K (Table 3)—this patient had high-grade disease (GS 8 at 66 yo) and admixed ancestry. Regarding the African ancestry-specific nature X285K, population MAF data from gnomAD show only a single occurrence of this variant out of 140,000 non-African-American individuals. The present inventors did find a different stop-loss mutation (p. X285SextX31, A to C c.854) in another EA patient with high-grade (GS 9) disease, at age 47.

Interestingly, putative founder variants have been seen in other ancestral populations, including Chinese and Japanese [19, 2. In both instances, like G84E, the recurrent variants found in these populations convert a Glycine codon to a Glutamic acid codon, although at two different positions: G132E in Japanese and G135E in Chinese. All three of these G to E variants are located close to or within conserved domains that all paralog group 13 HOX proteins share. These domains contain binding sites for the MEIS family of TALE homeobox cofactors, which are known to bind and modulate the transcriptional effects of HOXB13 [21, 2. While these observations suggest disrupted HOXB13-MEIS interactions may underlie the pro-carcinogenic activity of G to E-mutated HOXB13, this remains largely unexplored [22, 2. Furthermore, other than a more general dysregulation of HOXB13 function, whether these variant forms of HOXB13 share similar functional effects related to prostate carcinogenesis remains unknown.

HOXB13 G84E has been consistently shown to identify men at high risk for PCa in populations of men of European ancestry [7. Men carrying the G84E variant in HOXB13 have a significantly increased risk of PCa and are more likely to have a family history of positive, early-onset disease. To date, no other variant in HOXB 13 has been found to have similar clinical implications. This study suggests that X285K variant carriers in the AA population have a higher risk of clinically significant PCa and an earlier age of onset. If confirmed, these results would provide a rationale for incorporating this variant when screening AA men for PCa risk.

The limitations of this study are several fold: although over 1000 patients were studied, the number of variants observed is low, thus due to the rarity of these changes, the relatively small numbers of carriers in this study resulted in low power to perform additional subgroup analysis in multiple GS groups (eg≤6, =7 and ≥8), T stages, or other clinicopathologic variables. Further studies need to be conducted to assess and expand our study. It should also be noted that the interpretation of any results between cases and controls, using public datasets for the latter, should be done cautiously due to population heterogeneity and differing sequencing methods. In addition, the present inventors acknowledge the lack of generalizability of our observations to the general prostate cancer population overall due to the reliance on a hospital based, surgically treated study population. Other biases like those introduced by changes in screening and diagnostic practices, pathological grading over time, and referral patterns are possible, and could affect our results.

With respect to function, to make any causal inference beyond the associations from this study, mechanistic studies are necessary to understand how this variant might act to affect HOXB13 activity and promote PCa in AA. Finally, even if confirmed, the low population frequency indicates that the X285K likely accounts for a very small proportion of PCa disparities.

In summary, the present inventors describe the presence of a rare but recurrent germline variant in HOXB13 in AA men with PCa, and provide data to suggest a potentially important association with risk of early-onset, clinically significant PCa. Markers such as these, if validated, are urgently needed to provide useful risk stratification information for PCa in the high-risk AA population.

### Materials and Methods

### Study Subjects

This is a retrospective study including 1048 PCa patients of African-American ancestry as determined by self-report. Study subjects were patients undergoing radical prostatectomy (RP) for clinically localized PCa at the Brady Urological Institute of Johns

Hopkins Hospital Baltimore, Maryland, USA. Clinical and demographic information of these patients, including age at diagnosis, family history of PCa, surgical GS, and tumor staging (TMN) were obtained from an IRB approved, research database containing no PHI. The only criteria for selection, other than radical prostatectomy for PCa, was African-American ancestry and availability of discarded, deidentified, non-tumor involved tissue samples from surgery as a source of germline DNA. The institutional review board at Johns Hopkins University School of Medicine approved this study.

### Sequencing of Germline DNA and Bioinformatics Analysis.

Whole-exome sequencing was performed on germline DNA derived from non-tumor involved seminal vesicle tissue from the cases using Novogene sequencing service. The Agilent SureSelect Human All Exon V5 was used to capture and enrich exomic sequences. Enriched libraries were sequenced using an Illumina HiSeq 2500 system. The mean sequencing depth of coverage was 71x.

Paired-end reads were aligned to the GRCh37 version of the human genome using Burrows-Wheeler Aligner v0.7 to generate BAM files. After sorting the BAM files using samtools, PCR duplicates marked using Picard and realignment around putative gaps was performed using the Genome Analysis Toolkit (GATK) v3.2-2. Variant calling was performed with the GATK Haplotype caller. ANNOVAR (http://annovar.openbioinformatics.org/en/latest) and snpEff were used for annotating variants. For retrieving information including population frequency estimates, population-based databases ExAC (http://exac.broadinstitute.org/), and gnomAD (https://gnomad.broadinstitute.org) were used, and the clinical database, ClinVar (https://www.ncbi.nlm.nih.gov/clinvar/variation/) was used to assess the pathogenicity of variants [1. SIFT (https://sift.bii.a-star.edu.sg) and Polyphen2 (http://genetics.bwh.harvard.edu/pph2/) were used to assess potential deleteriousness of non-synonymous changes.

### Statistical Analysis.

The frequency of variants in HOXB13 was compared and analyzed using Fisher's exact test. A proportional trend test was used to estimate the significance of trends among multiple groups.

The present inventors used 2170 ancestral informative markers (AIMs) of the Illumina global screening array (GSA) to assess genetic background of self-reported African-American (AA) subjects. The analysis was performed using principal component analysis within PLINK and the top 20 principal components (eigens) were obtained [1. The present inventors also plotted the top two eigens of AA subjects together with three anchored racial populations for 1 KG (CEU, YRI and EAS). All AA subjects in our study are consistent with recent admixture of CEU and YRI [1.

A type I error of 0.05 (two-sided) was used to define statistical significance. All the statistical analyses were performed using R software (version 4.0.4). The OR and 95% confidence interval were estimated using the R software “fisher.test”. All ORs were adjusted for principal component eigens to account for potential confounding by ancestry.

TABLE 1
Baseline demographic characteristics of the study population

| | | Clinical variables by Gleason Score | | |
|---|---|---|---|---|
| Variables | All | GS ≥ 7 | GS ≤ 6 | P value |
| No. of patients | 1048 | 650 (62.0%) | 397 (37.9%) | — |
| Age at diagnosis (year, median + IQR) | 57 (52-62) | 58 (52-63) | 55 (51-60) | 1.58 × 10⁻⁸ |
| PSA (mean ± SD) | 7.64 ± 6.32 | 8.73 ± 7.31 | 5.87 ± 3.61 | <2.20 × 10⁻¹⁶ |

Gleason Score (n, %)

| | | | | |
|---|---|---|---|---|
| <=6 | 397 (37.9%) | 0 (0.00%) | 397 (100%) | — |
| 7 | 542 (51.7%) | 542 (83.4%) | 0 (0.00%) | |
| >=8 | 108 (10.3%) | 108 (16.6%) | 0 (0.00%) | |
| Missing | 1 (0.1%) | — | — | |

Family history**

| | | | | |
|---|---|---|---|---|
| Positive | 493 (47.0%) | 305 (46.9%) | 187 (47.1%) | 0.77 |
| Negative | 528 (50.4%) | 333 (51.2%) | 195 (49.1%) | |
| Missing | 27 (2.6%) | 12 (1.8%) | 15 (3.8%) | |

Pathological TMN stage

| | | | | |
|---|---|---|---|---|
| T₂N₀(Nₓ)Mₓ | 700 (66.8%) | 364 (56.0%) | 336 (84.6%) | <2.20 × 10⁻¹⁶* |
| T₃ₐN₀(Nₓ)Mₓ | 208 (19.8%) | 161 (24.8%) | 47 (11.8%) | |
| T_{3b}N₀(Nₓ)Mₓ | 76 (7.3%) | 67 (10.3%) | 4 (1.0%) | |
| All stages N₁ | 32 (3.1%) | 32 (4.9%) | 0 (0.0%) | |
| TₓNₓMₓ/missing | 32 (3.1%) | — | — | |

*Proportion trend test.
**First or second-degree relative diagnosed with prostate cancer.

TABLE 2
Non-synonymous variants in HOXB13 (chr17q21.32, Access No. NM_006361) in AA PCa cases

| | POS | | | | | | | |
|---|---|---|---|---|---|---|---|---|
| CHR | (hg19) | Type | Exon | CDNA | Protein | MAF | SIFT | Polyphen2 |
| 17 | 46804154 | Stoploss | 2 | c.853delT | p.X285K | 0.0038 | na | na |
| 17 | 46804282 | Missense | 2 | c.C725T | p.T242I | 0.0005 | Deleterious | Possibly damaging |
| 17 | 46805555 | Missense | 1 | c.C401A | p.P134Q | 0.0005 | Tolerated | Possibly damaging |
| 17 | 46805639 | Missense | 1 | c.T317G | p.L106R | 0.0005 | Tolerated | Possibly damaging |
| 17 | 46805657 | Missense | 1 | c.G299A | p.C100Y | 0.0005 | Deleterious | Benign |
| 17 | 46805679 | Missense | 1 | c.T277G | p.S93A | 0.0005 | Tolerated | Benign |
| 17 | 46805705 | Missense | 1 | c.G251A | p.G84E | 0.0005 | Deleterious | Probably damaging |

MAF minor allele frequency in study population, AFR African American, NFE non-Finnish European.

TABLE 3
Clinicopathologic information for X285K carriers

| | Pre-biopsy | Age at | | Pathology | | | | | | | | | | |
|---|---|---|---|---|---|---|---|---|---|---|---|---|---|---|
| ID | PSA (ng/ml) | diagnosis | Ancestry | Gleason Score | | FH | pr wt | T | FCF CP | E ECP | SM | OC | LN | SV |
| 1 | 5.1 | 64 | African- | 7 | 4 + 3 | 0 | 37.6 | T3a | N | Y | N | N | N | N |
| | | | American | | | | | | | | | | | |
| 2 | 11.6 | 41 | African- | 7 | 3 + 4 | 0 | 35.7 | T2 | N | N | Y | Y | N | N |
| | | | American | | | | | | | | | | | |
| 3 | 5.8 | 60 | African- | 7 | 3 + 4 | 1 | 28.9 | T2 | N | N | N | Y | N | N |
| | | | American | | | | | | | | | | | |
| 4 | 2.4 | 45 | African- | 7 | 3 + 4 | 1 | 33.1 | T2 | N | N | N | Y | N | N |
| | | | American | | | | | | | | | | | |
| 5 | 18 | 49 | African- | 7 | 3 + 4 | 0 | 47.5 | T3b | Y | N | N | N | Y | Y |
| | | | American | | | | | | | | | | | |
| 6 | 5 | 51 | African- | 9 | 4 + 5 | 0 | 33.9 | T2 | N | N | N | Y | N | N |
| | | | American | | | | | | | | | | | |
| 7 | 13 | 65 | African- | 7 | 4 + 3 | 0 | 73.7 | T2 | N | N | N | Y | N | N |
| | | | American | | | | | | | | | | | |
| 8 | 7.4 | 39 | African- | 7 | 4 + 3 | 0 | 41.2 | T2 | N | N | N | Y | N | N |
| | | | American | | | | | | | | | | | |
| 9* | 5.1 | 66 | European- | 8 | 3 + 5 | 0 | 61.4 | T3a | N | Y | Y | N | N | N |
| | | | American | | | | | | | | | | | |

T surgical T stage, FCP focal capsular penetration, ECP extensive capsular penetration, SM surgical margins, OC organ confined, LN lymph node positive, SV seminal vesicle invasion, FH first or second-degree relative with PCa, pr wt prostate weight, Y yes/positive, N no/negative, na not available.
*This patient is a self-reported European American with African and European admixture in the ancestry analysis.

TABLE 4
Carrier rates of the HOXB13 mutation (c.853delT X285K)
in AA population and in different GS groups

| | | Clinical significance based on Gleason Score | | | |
|---|---|---|---|---|---|
| | | GS ≥ 7 | GS ≤ 6 | | |
| Mutation | Carrier rates | (n = 650) | (n = 397) | OR (95% CI) | P value |
| Negative | 1034 (98.7%) | 638 (98.2%) | 395 (99.5%) | Ref | Ref |
| c.853delT | 8 (0.8%) | 8 (1.2%) | 0 (0%) | inf (1.05-inf) | 0.028 |
| X285K | | | | | |

TABLE 5
Carrier rates of the HOXB13 mutation, c.853delT X285K,
in AA populations with different ages at diagnosis

| | Age at diagnosis | | | |
|---|---|---|---|---|
| | PCa < 50 year | PCa ≥ 50 year | | |
| Mutation | (n = 170) | (n = 878) | OR (95% CI) | P value |
| Negative | 165 (97.1%) | 869 (99.0%) | Ref | Ref |
| c.853delT | 4 (2.4%) | 4 (0.5%) | 5.25 (1.00-28.52) | 0.03 |
| X285K | | | | |

### REFERENCES

1. American Cancer Society. Cancer Facts & Figures for African Americans 2019-2021. 2019. https://www.cancer.org/content/dam/cancer-org/research/cancerfacts-and-statistics/cancer-facts-and-figures-for-african-americans/cancer-factsand-figures-for-african-americans-2019-2021.pdf (accessed 16 Mar. 2021).
2. Paller C J, Wang L, Brawley O W. Racial inequality in prostate cancer outcomessocioeconomics, not biology. JAMA Oncol. 2019; 5:983-4.
3. Abeshouse A, Ahn J, Akbani R, Ally A, Amin S, Andry C D, et al. The molecular taxonomy of primary prostate cancer. Cell. 2015; 163:1011-25.
4. Ewing C M, Ray A M, Lange E M, Zuhlke K A, Robbins C M, Tembe W D, et al. Germline mutations in HOXB13 and prostate-cancer risk. N. Engl J Med. 2012; 366:141-9.
5. Nyberg T, Govindasami K, Leslie G, Dadaev T, Bancroft E, Ni Raghallaigh H, et al. Homeobox B13 G84E mutations and prostate cancer risk. Eur Urol. 2019; 75:834-45.
6. Xu J, Lange E M, Lu L, Zheng S L, Wang Z, Thibodeau S N, et al. HOXB13 is a susceptibility gene for prostate cancer: results from the International Consortium for Prostate Cancer Genetics (ICPCG). Hum Genet. 2013; 132:5-14.

7 Karlsson R, Aly M, Clements M, Zheng L, Adolfsson J, Xu J, et al. A populationbased assessment of germline HOXB13 G84E mutation and prostate cancer risk. Eur Urol. 2014; 65:169-76.

8 Laitinen V H, Wahlfors T, Saaristo L, Rantapero T, Pelttari L M, Kilpivaara O, et al. HOXB13 G84E mutations in Finland: population-based analysis of prostate, breast, and colorectal cancer risk. Cancer Epidemiol Biomark Prev. 2013; 22:452-60.
9 Akbari M R, Trachtenberg J, Lee J, Tam S, Bristow R, Loblaw A, et al. Association between germline HOXB13 G84E mutations and risk of prostate cancer. J Natl Cancer Inst. 2012; 104:1260-2.
10. Marlin R, Creoff M, Merle S, Jean-Marie-Flore M, Rose M, Malsa S, et al. Mutation HOXB13 c.853delT in Martinican prostate cancer patients. Prostate. 2020; 80:463-70.
11. Landrum M J, Lee J M, Riley G R, Jang W, Rubinstein W S, Church D M, et al. ClinVar: public archive of relationships among sequence variation and human phenotype. Nucleic Acids Res. 2014; 42. https://doi.org/10.1093/nar/gkt1113.
12. Galinsky K J, Bhatia G, Loh P R, Georgiev S, Mukherjee S, Patterson N J, et al. Fast principal-component analysis reveals convergent evolution of ADHIB in Europe and East Asia. Am J Hum Genet. 2016; 98:456-72.
13. Economides K D, Capecchi M R. Hoxb13 is required for normal differentiation and secretory function of the ventral prostate. Development. 2003; 130:2061-9.
14. Pomerantz M M, Li F, Takeda D Y, Lenci R, Chonkar A, Chabot M, et al. The androgen receptor cistrome is extensively reprogrammed in human prostate tumorigenesis. Nat Genet. 2015; 47:1346-51.
15. Chen Z, Wu D, Thomas-Ahner J M, Lu C, Zhao P, Zhang Q, et al. Diverse AR-V7 cistromes in castration-resistant prostate cancer are governed by HoxB13. Proc Natl Acad Sci USA. 2018; 115:6810-5.
16. Norris J D, Chang C Y, Wittmann B M, Kunder R S, Cui H, Fan D, et al. The homeodomain protein HOXB13 regulates the cellular response to androgens. Mol Cell. 2009; 36:405-16.
17. Witte J S, Mefford J, Plummer S J, Liu J, Cheng I, Klein E A, et al. HOXB13 mutation and prostate cancer: Studies of siblings and aggressive disease. Cancer Epidemiol Biomark Prev. 2013; 22:675-80.
18. Dhamija S, Yang C M, Seiler J, Myacheva K, Caudron-Herger M, Wieland A, et al. A pan-cancer analysis reveals nonstop extension mutations causing SMAD4 tumor suppressor degradation. Nat Cell Biol. 2020; 22:999-1010.
19. Momozawa Y, Iwasaki Y, Hirata M, Liu X, Kamatani Y, Takahashi A, et al. Germline pathogenic variants in 7636 Japanese patients with prostate cancer and 12 366 controls. J Natl Cancer Inst. 2020; 112:369-76.
20. Lin X, Qu L, Chen Z, Xu C, Ye D, Shao Q, et al. A novel germline mutation in HOXB13 is associated with prostate cancer risk in Chinese men. Prostate. 2013; 73:169-75.
21. Williams T M, Williams M E, Innis J W. Range of HOX/TALE superclass associations and protein domain requirements for HOXA13: MEIS interaction. Dev Biol. 2005; 277:457-71.
22. Bhanvadia R R, Van Opstall C, Brechka H, Barashi N S, Gillard M, McAuley E M, et al. MEIS1 and MEIS2 expression and prostate cancer progression: a role for HOXB13 binding partners in metastatic disease. Clin Cancer Res. 2018; 24:3668-80.
23. Johng D, Torga G, Ewing C M, Jin K, Norris J D, McDonnell D P, et al. HOXB13 interaction with MEIS1 modifies proliferation and gene expression in prostate cancer. Prostate. 2019; 79:414-24.

### Example 2: Clinical and Functional Analyses of an African-Ancestry Gain-of-Function HOXB13 Variant Implicated in Aggressive Prostate Cancer

### Background.

Recent reports have uncovered a HOXB13 variant (X285K) predisposing to prostate cancer in men of West-African ancestry. The clinical relevance and protein function associated with this inherited variant is unknown.

### Objective.

To determine the clinical relevance of HOXB13 (X285K) in comparison with HOXB13 (G84E) and BRCA2 pathogenic/likely pathogenic (P/LP) variants, and to elucidate the oncogenic mechanisms of the X285K protein.

### Design, Setting, and Participants.

Real-world data from 21,393 men with prostate cancer undergoing genetic testing from 2019-2022, and in vitro cell-line models for evaluation of oncogenic functions associated with the X285K protein.

### Outcome Measurements and Statistical Analysis.

Genetic testing results were compared among patient groups according to self-reported race/ethnicity, Gleason scores, and AJCC stages using exact test. Oncogenic functions of X285K were evaluated by RNA sequencing, ChIP-sequencing, and Western blot analyses.

### Results and Limitations.

HOXB13 (X285K) was significantly enriched in self-reported Black (1.01%) versus White (0.01%) patients. We observed a trend of more aggressive disease in the HOXB13 (X285K) and BRCA2 P/LP carriers than in the HOXB13 (G84E) carriers. Replacement of the wild-type (WT) HOXB13 protein with the X285K protein resulted in a gain of an E2F/MYC signature, validated by a gain in the expression of Cyclin B1 and c-MYC, without affecting the androgen response signature. Elevated expression of Cyclin B1 and c-MYC was explained by enhanced binding of the X285K protein to the promoters and enhancers of these genes. The limitations of the study are the lack of complete clinical outcome data for all patients studied and the use of a single cell line in the functional analysis.

### Conclusions.

HOXB13 X285K is significantly enriched in self-reported Black patients and X285K carriers detected in the real-world clinical setting have aggressive prostate cancer features similar to the BRCA2 carriers. Functional studies revealed a unique gain-of-function oncogenic mechanism of X285K protein in regulating E2F/MYC signatures.

### Patient Summary.

The HOXB13 X285K variant is clinically and functionally linked to aggressive prostate cancer, supporting early disease screening of Black men carrying the HOXB13 X285K variant.

### Introduction

Heritable genetic risk factors play a significant role in prostate cancer etiology and clinical practice [1]. Currently, the adoption of genetic testing in prostate cancer is mainly driven by the established prognostic and treatment utility of rare pathogenic variants in BRCA2 and other DNA damage response (DDR) pathway genes [2]. The underlying pathogenic mechanism for these variants is loss of DNA repair function, often due to frameshift or premature stop alterations causing protein truncation [3]. In addition to DDR genes, multiple genetic variants associated with prostate cancer risk have been found in the HOXB13 gene [4,5] in different ancestral populations, including European [6] (e.g., G84E), Japanese (G132E) [7], and Chinese (G135E) [8]. Unlike pathogenic DDR gene variants, these HOXB13 variants result in alterations in a single amino acid, and, while useful for early disease screening, there is little evidence supporting their prognostic and treatment utility. The functional implications of germline HOXB13 variants detected in European and East Asian ancestral populations remain poorly characterized, although the wild-type HOXB13 protein is known to be abundantly and specifically expressed in cells of prostatic lineage [4,9] with roles in regulating the androgen receptor axis [10, 11].

Recently, a stop-loss HOXB13 variant c.853delT (referred to as X285K), in which a single base deletion within the HOXB13 stop codon (c.853delT) results in an extension of the HOXB13 protein by 96 amino acids, was identified as a risk factor for prostate cancer in men of African ancestry [12-14]. This variant was first reported to be associated with prostate cancer by Martin et al. in 3 prostate cancer patients in Martinique out of a total of 46 prostate cancer patients diagnosed before age 51 [13]. Subsequently, we reported eight X285K carriers identified by whole-exome sequencing in a cohort of 1048 self-reported African American prostate cancer patients who had radical prostatectomy [14]. All eight carriers had prostate cancers of Gleason grade 7-9. Most recently, in Darst et al. [12], X285K carrier status was determined by genetic imputation and associated with late-stage disease, with an overall carrier rate of 0.7% in prostate cancer patients of African ancestry. It was estimated that the variant emerged ~1500-4600 years ago in West Africa [12]. Therefore, HOXB13 X285K represents a unique germline variant affecting the risk for aggressive prostate cancer specifically in men of African ancestry.

These recent reports raise important questions about the clinical relevance of HOXB13 (X285K) in the context of other clinically established prostate cancer genetic risk factors (e.g., BRCA2). In addition, there is a critical need to understand whether and how the X285K protein changes the HOXB 13 function. Here, we present real-world germline genetic testing results and compare clinical characteristics between patients with HOXB13 variants (X285K and G84E) and BRCA2 pathogenic/likely pathogenic (P/LP) variants. We performed in vitro functional analyses comparing the HOXB13 (X285K) variant with its wild-type (WT) counterpart, revealing a unique gain-of-function oncogenic mechanism related to E2F/MYC signaling. These findings inform genetic testing strategies and potential therapeutic development targeting HOXB13 (X285K).

### Materials and Methods

### Germline Genetic Testing Data Analysis.

We conducted an IRB-approved (protocol 1167406) review of de-identified data from 21,393 men with prostate cancer who received clinical-grade germline testing as previously described [15]. Patients included in this analysis were tested through the Detect Hereditary Prostate Cancer (DHPC) sponsored (no-charge) testing program which ran from 2019-2022 (Invitae Corporation, San Francisco, CA). Inclusion criteria for DHPC were low, intermediate or high-risk localized, stage>IIb disease, or stage IIA disease diagnosed <55 years old. Briefly, patients underwent full gene sequencing, including deletion-duplication analysis for cancer-relevant genes, with the number of genes ordered varying at the discretion of the ordering clinician. Variants were classified using a refinement of the American College of Genetics and Genomic criteria (Invitae's® Sherloc) [16], and those classified as pathogenic or likely pathogenic (P/LP) were orthogonally confirmed. Patients who underwent testing for HOXB13 (G84E and X285K) variants (n=21,091) or BRCA2 P/LP variants (n=21,362) were stratified by self-reported race/ethnicity: Black (or African-American), White (non-Hispanic), and all other (including multiracial) groups. Gleason grades and AJCC tumor stage were clinician-reported on the genetic testing requisition form. Statistical analyses were completed using R (version 4.1.2) and a significance threshold of α<0.05 was used. Additional details are described in the Supplemental Appendix.

### Functional Analysis.

Detailed in vitro functional analyses were conducted in stable LNCaP95 (LN95) cell clones that were engineered to express exogenous HOXB13 WT (n=5) and HOXB13 X285K (n=3) proteins (hereafter called LN95WT and LN95X285K, respectively) upon induction by doxycycline (Dox). By design, treatment with Dox simultaneously induces the knockdown of endogenous WT HOXB13 (Supplementary Appendix). As a control, three clones carrying non-targeting (NT) control vectors were generated (hereafter called LN95NT). These clones (n=11) were subjected to RNA sequencing (RNA-Seq), protein analyses, and chromatin immunoprecipitation (ChIP) sequencing (ChIP-Seq). Details are provided in the Supplementary Appendix.

### Results

Germline Genetic Testing Results from the DHPC Program.

To determine the clinical relevance of HOXB13 (X285K) in real-world practice, we extracted genetic testing data from 21,393 patients and interrogated for HOXB13 (X285K), together with HOXB13 (G84E), as well as established BRCA2 P/L P variants. Notably, while the prevalence of HOXB13 (X285K) in prostate cancer patients was 0.18% overall, this was markedly enriched in men of self-reported Black versus White ancestry (1.01% vs. 0.01%) (Table 6). Conversely, the HOXB13 (G84E) variant was enriched in White versus Black patients (1.12% vs. 0.11%). By comparison, the BRCA2 P/LP rate in this real-world setting was 1.58% and 1.92%, respectively, in self-reported White and Black patients (Table 6). Further, we demonstrated differences in Gleason scores (p=2.8×10⁻⁶) and cancer stages (p=6.6×10⁻⁴) among HOXB13 (X285K) carriers, HOXB13 (G84E) carriers, and BRCA2 carriers (Table 6). Compared to G84E carriers, X285K and BRCA2 P/LP carriers had a higher prevalence of Stage IV (including metastatic disease) at presentation (93.7% vs. 67.7%, p=0.037 and 85.6% vs. 67.7%, p=2.5×10⁻⁴, respectively). Although statistically insignificant, compared to G84E carriers, X285K carriers also had a numerically higher prevalence of Gleason 8-10 histology (62.5% vs 49.3%, p=0.243). Interestingly, we did not detect differences between X285K carriers and BRCA2 P/LP carriers in cases with Gleason 8-10 histology (62.5% vs. 70.0%, p=0.423) and Stage IV disease at presentation (93.7% vs. 85.6%, p=0.707), though, within the Gleason 8-10 histology, numerical differences in Gleason 8 (40.6% vs. 20.5%) and Gleason 9-10 (21.9% vs. 49.5%) prevalence were observed between the X285K carriers versus BRCA2 P/LP carriers (Table 6). Among the 2715 self-reported Black patients (2680 tested for HOXB13), a “non-carrier” group (n=2626) was created after excluding BRCA2 (n=52), BRCA1 (n=7), X285K (n=27), and G84E (n=3) carriers (Table 8). Among Black patients, we found a numerically higher prevalence of Gleason 8-10 histology (63.6% vs. 52.2%, p=0.39) and Stage IV disease (91.7% vs. 77.1%, p=0.32) in the X285K carriers, when compared to the “non-carrier” group.

### Population Attributable Risk.

Results from the real-world genetic testing data are limited by the lack of a control population. Nevertheless, population-attributable risk (PAR) can be estimated for each specific genetic variant. PAR for HOXB13 (X285K) was estimated to be 0.62% in the Black/African-American population (Table 7). By comparison, PAR was 0.59% for HOXB13 (G84E) in the white population and 1.19% for BRCA2 in the entire population (Table 7).

### Establishment of In Vitro Models for X285K.

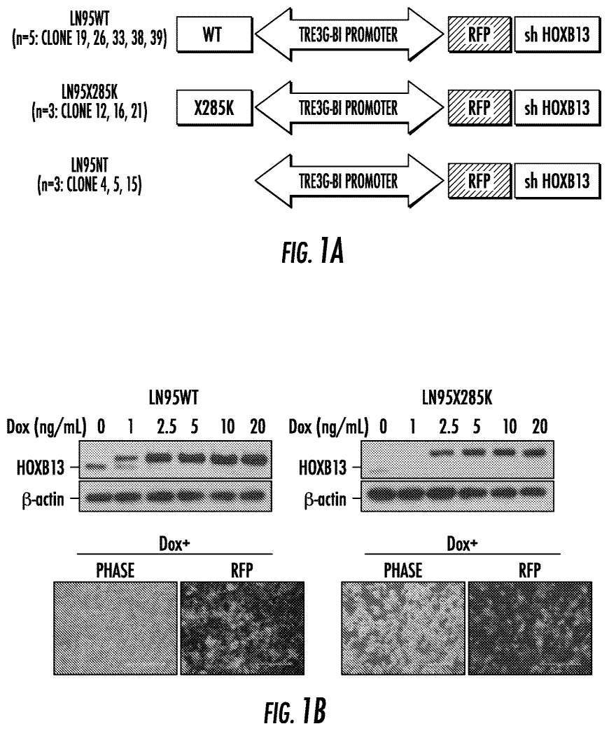

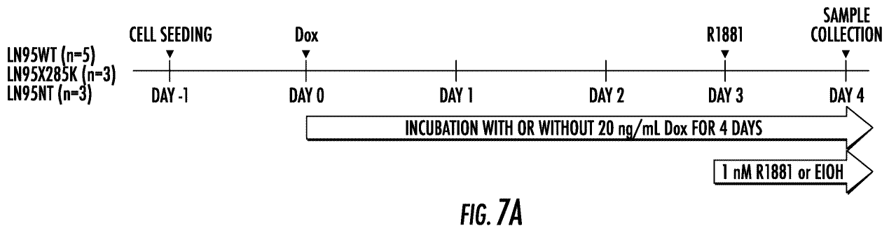

Structurally, X285K is a single-base deletion within the HOXB13 stop codon (c.853delT), resulting in an extension by 96 amino acids of the HOXB13 protein (p. Ter285Lys ext96) at the C-terminal of the DNA-binding homeobox domain as visualized by Alphafold (FIG. 5). To study the functions mediated by X285K, we devised a strategy (FIG. 1A) to replace the WT HOXB13 with X285K in LNCaP95, a castration-resistant prostate cancer (CRPC) cell line demonstrating HOXB13-dependent AR/AR-V7 functional output [17, 18]. Treatment with increasing doses of doxycycline (Dox) successfully displaced the endogenous WT HOXB13 with exogenous WT or X285K HOXB13 in a dose-dependent manner (FIG. 1B). Replacement of endogenous WT with exogenous X285 was validated by both Western blot analyses and qRT-PCR in all 11 stable clones following five different Dox doses (FIG. 6).

### RNA-Sequencing Analyses.

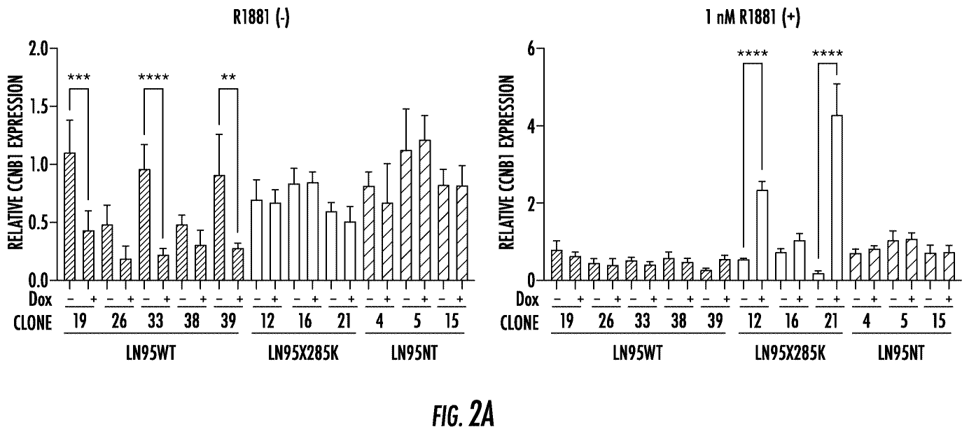

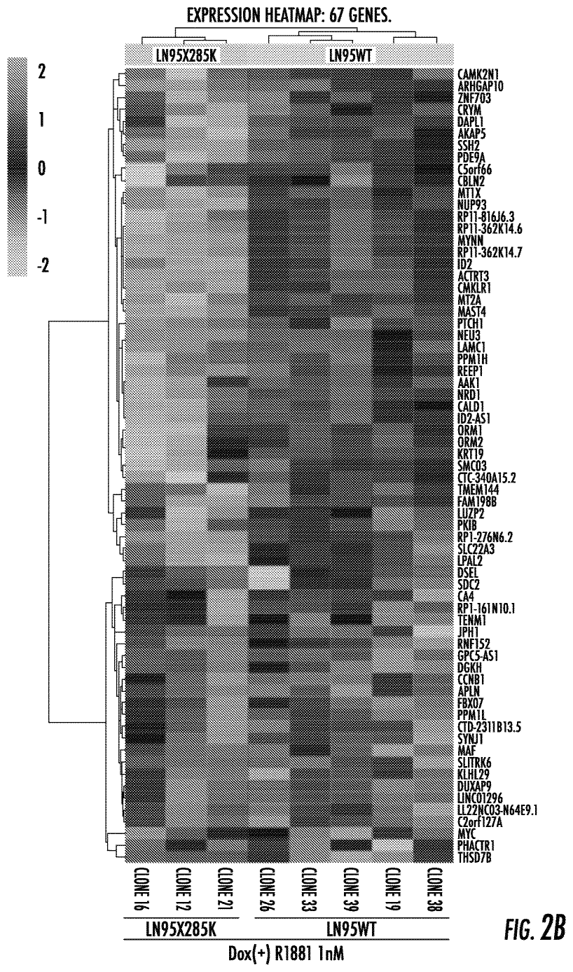

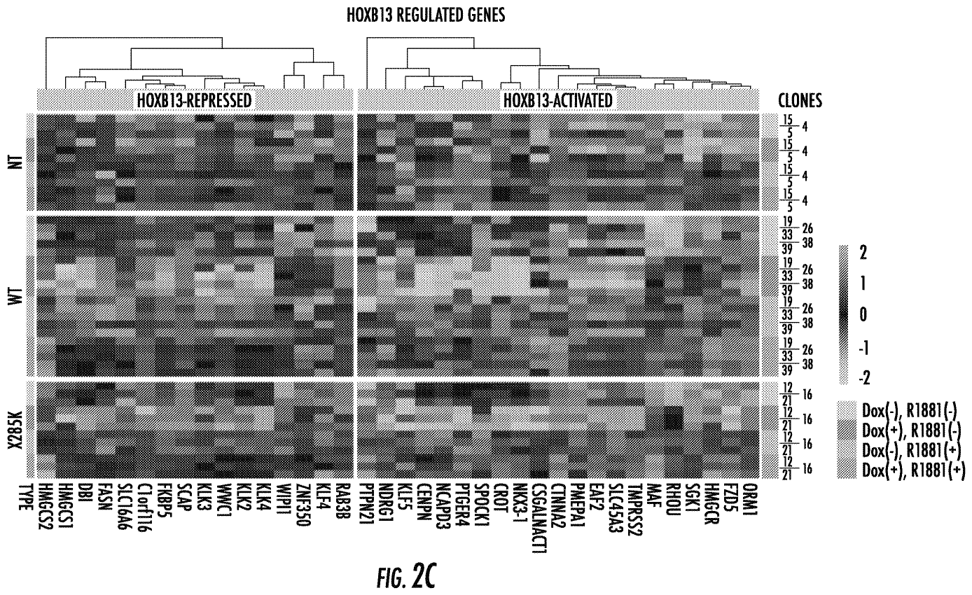

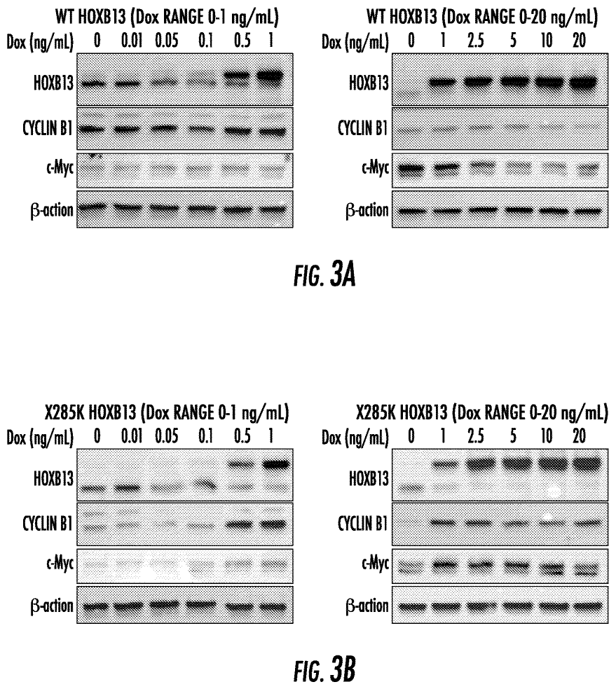

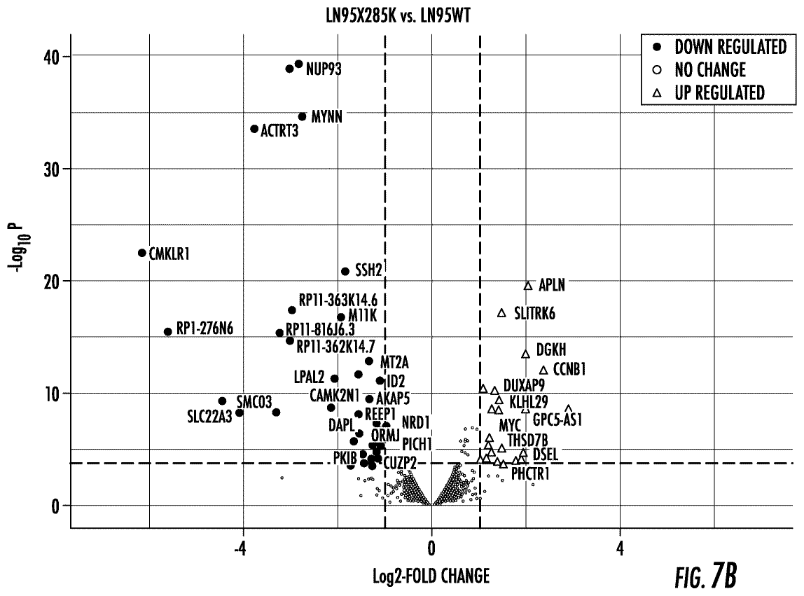

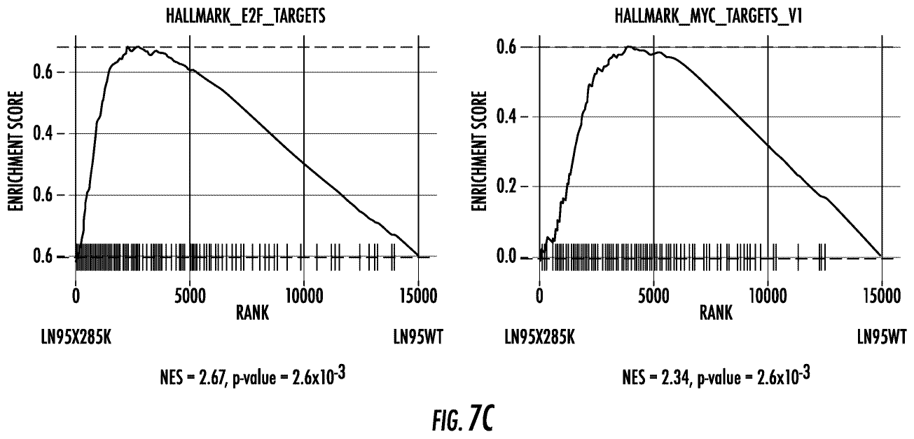

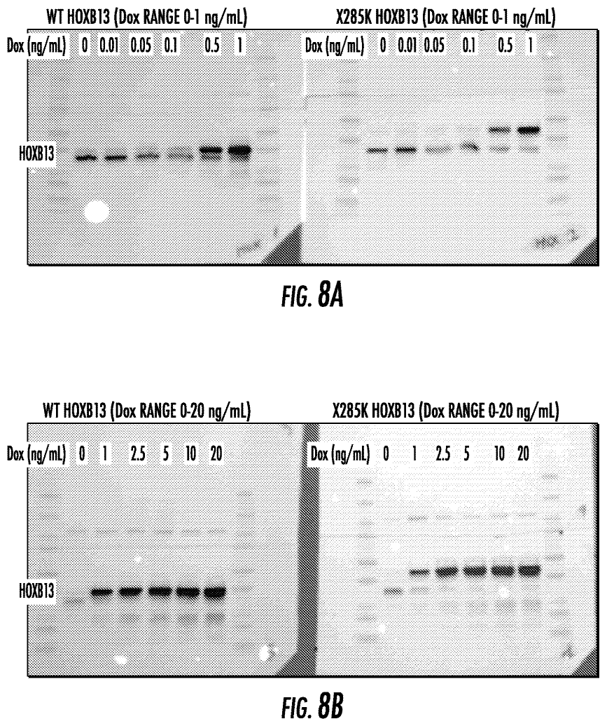

We generated RNA-sequencing (RNA-Seq) data from 44 cell line samples after subjecting each of the 11 stable clones (five LN95WT, three LN95X285K, and three LN95NT) to 4 different treatment conditions (with or without Dox, in the presence or absence of androgen) (FIG. 7A). Examination of the sequencing data did not uncover a change in AR/AR-V7 expression (not shown). Surprisingly, CCNB1 (encoding cyclin B1) was identified as a top-ranked gene that was upregulated following induction of exogenous X285K by Dox treatment (FIG. 2A, RT-PCR validation of RNA-Seq data). Interestingly, this occurred only in androgen-stimulated conditions (FIG. 2A), and the degree of CCNB1 upregulation appeared to positively correlate with the abundance of exogenous X285K expression (FIG. 6). Conversely, overexpression of WT HOXB13 suppressed CCNB1 expression (FIG. 2A). We next conducted unbiased analyses focusing on the comparison of WT and X285K in androgen-stimulated cells. Notably, differential gene expression analysis identified MYC and CCNBI among genes significantly upregulated in X285K clones compared with WT clones (FIG. 2B, FIG. 7B). In addition, gene set enrichment analysis revealed E2F and MYC targets as the top gene sets enriched in X285K clones (FIG. 7C).

Because HOXB13 interacts with AR, a key therapeutic target in prostate cancer, we performed the analysis of a previously published set of HOXB 13-activated and HOXB13-repressed AR target genes to determine whether HOXB13 WT and X285K demonstrate differential functions (i.e., functional gain or loss) either as activators or repressors of AR target genes. Overexpression of both exogenous WT and X285K HOXB13 resulted in suppression of HOXB 13-repressed genes in both androgen-deprived and androgen-stimulated conditions, confirming the largely intact AR repressor function of both WT and X285K (FIG. 2C). Similarly, HOXB 13-activated genes remain equally inducible upon androgen stimulation in cells with either endogenous WT, exogenous WT, or exogenous X285K (FIG. 2C). These results are consistent with the previously reported bifunctional activity of HOXB13 and suggest that the function of X285K with respect to regulating multiple AR target genes is virtually indistinguishable from WT.

### Validation of Gain-of-Function by Western Blot Analyses.

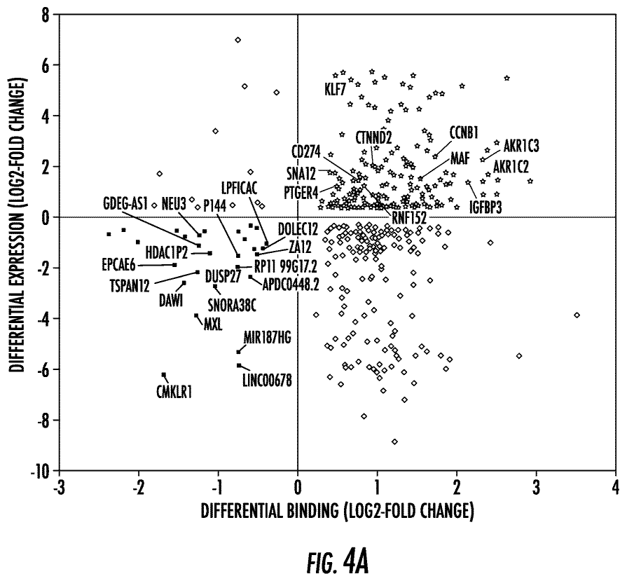

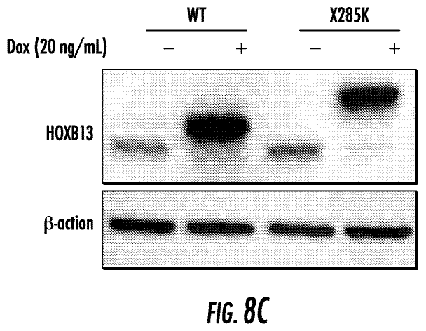

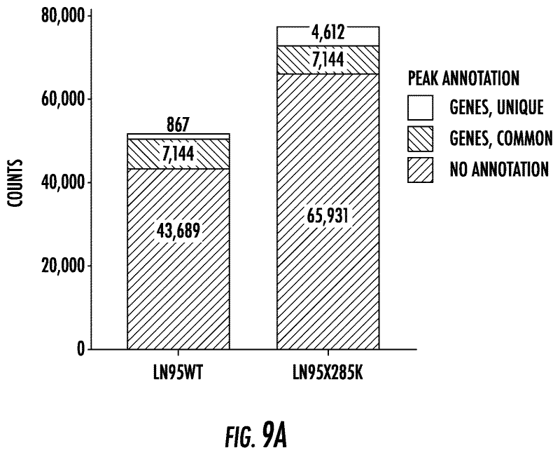

To validate a gain-of-function mechanism in regulating E2F/MYC, we conducted Western blot analyses to evaluate CCNB1 and MYC protein expression following the induction of varying levels of X285K. Consistent with the RNA-Seq data, we detected a Dox dose-dependent increase of CCNB1 and MYC protein expression in X285K clones. By contrast, a dose-dependent decrease in CCNB1 and MYC was observed in WT clones (FIGS. 3A and 3B, FIG. 8A-9C).

### Validation of Gain-of-Function by ChIP Sequencing.

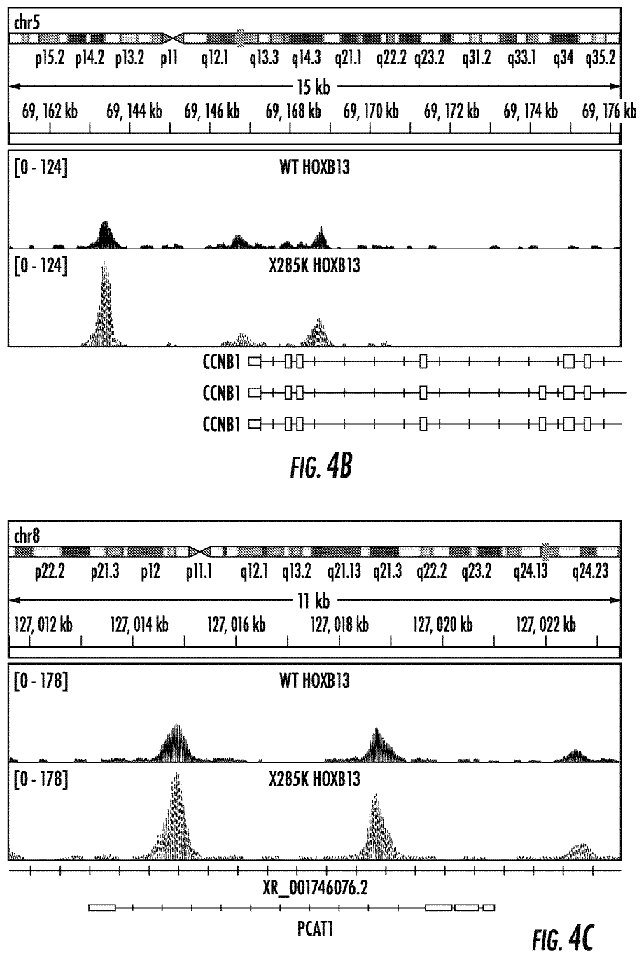

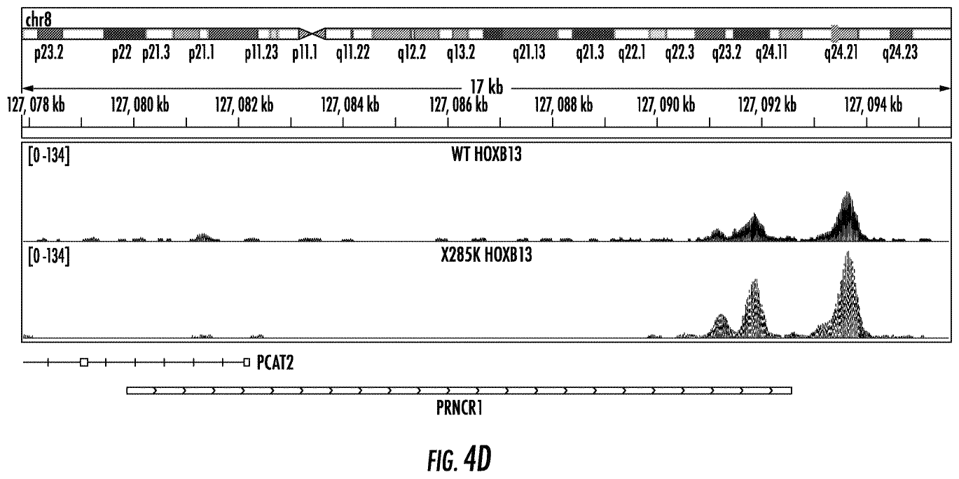

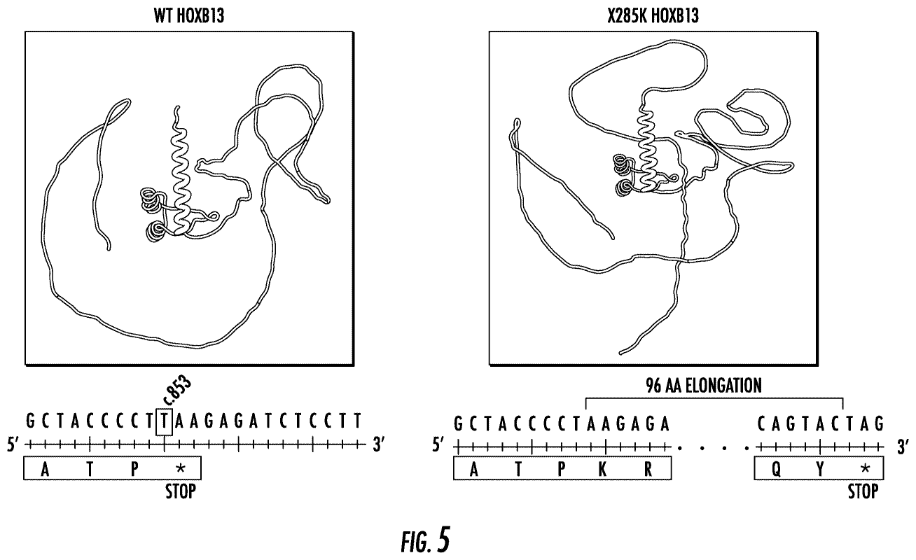

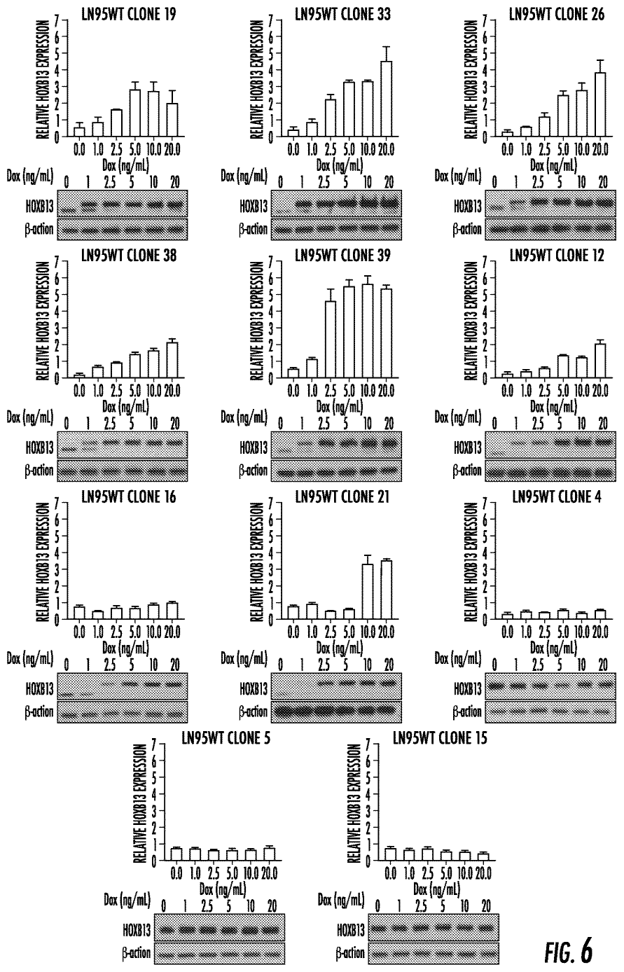

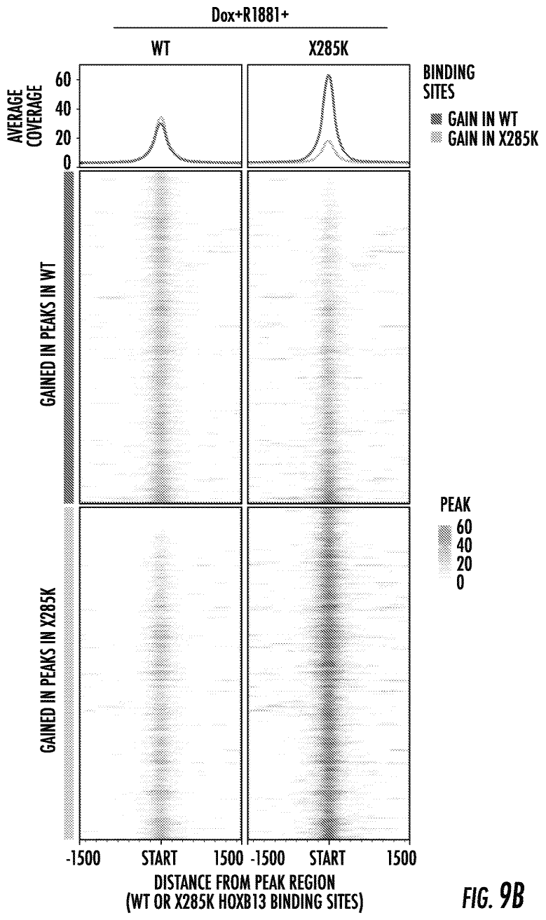

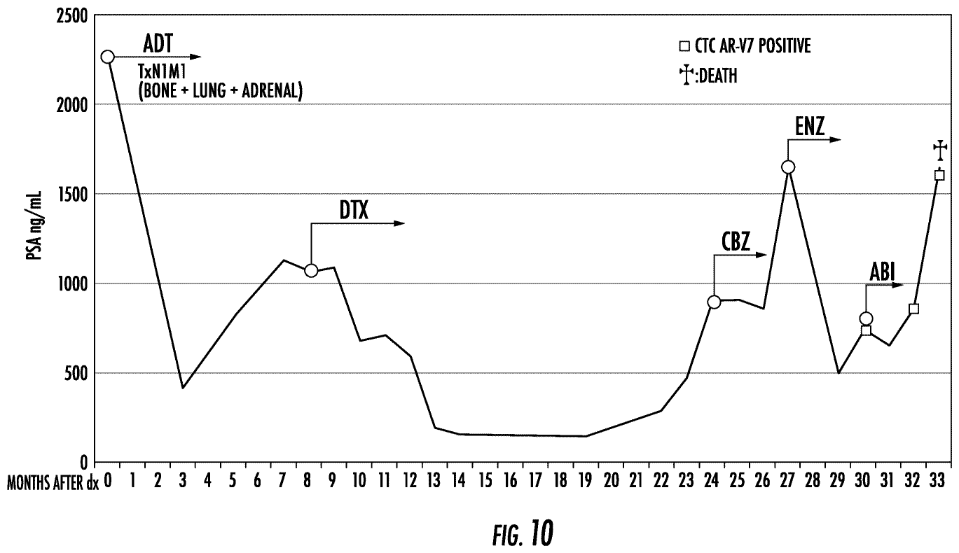

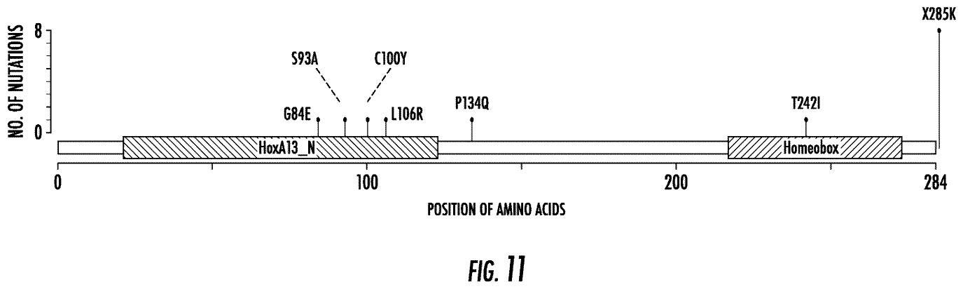

Next, we performed HOXB13 ChIP-Seq to determine genomic binding sites that differ between the WT and X285K HOXB13 proteins. Notably, ChIP-Seq detected 4612 X285K-specific, annotated binding sites (FIG. 9A). In comparison, only 867 binding sites were specific to the WT protein. In addition, the X285K-specific binding sites demonstrated substantially higher peaks, suggesting global epigenetic changes that are specifically induced by X285K (FIG. 9B). To determine if this differential binding pattern affects gene expression, we conducted combined RNA-Seq and ChIP-Seq analysis. Interestingly, CCNB1 was one of the genes showing elevated gene expression as well as X285K binding (FIG. 4A). We confirmed a higher enrichment of X285K at the 5′ upstream region of CCNB1 (FIG. 4B), suggesting X285K elevates CCNB1 expression by direct binding. Next, we confirmed increased X285K binding to PCAT1 and PCAT2 regions (FIGS. 4C and 4D). PCAT1 and PCAT2 gene loci have recently been reported as MYC super-enhancers [19], suggesting that increased X285K binding to these regions contributes to elevated MYC expression and more aggressive clinical phenotypes.

### Discussion

The current study characterizing the clinical and functional relevance of the HOXB13 (X285K) variant was motivated by three recent publications implicating its association with aggressive prostate cancer in men of African ancestry [12-14]. We recognized that while the three studies yielded largely concordant findings, they were conducted in genetic research settings that may not reflect real-world practice settings. In addition, these single-variant studies did not capture the breadth of various rare germline variants with respect to their detection rates and associated patient characteristics. Additionally, these studies did not address whether and how the X285K protein alters the HOXB13 functional output. Lack of functional evidence demonstrating the impact on biological processes and pathways will impede variant classification and clinical translation. The current study was also motivated by the relevance to prostate cancer disparities [20, 21]. While it is well-conceived that socioeconomic determinants of prostate cancer disparity impact disease presentation, including clinical, pathological, and molecular characteristics [21, 22], it is also recognized that genetic variants contribute to population differences in risk for prostate cancer [23]. In this regard, it would be necessary to conduct clinical and functional characterization of the ancestry-specific HOXB13 X285K variant in order to understand the disease etiology and develop clinical utility in a patient population disproportionately affected by aggressive prostate cancer.

Our findings from the analysis of real-world genetic testing data are noteworthy and clinically informative in several aspects. First, we found that the X285K variant is detected in 1.01% of self-reported Black/African American prostate cancer patients undergoing genetic testing. This detection rate is nearly equivalent to the 1.12% detection rate for the G84E variant in self-reported White patients. In comparison to the G84E carriers, X285K carriers were enriched for prostate cancers with Gleason score 8 and AJCC Stage IV diseases, whereas BRCA2 tumors are enriched for Gleason 9-10 and AJCC Stage IV diseases. G84E is the most common HOXB13 variant in men of European ancestry. This variant was reproducibly associated with the risk of prostate cancer incidence [6], but the association was equally strong in men with aggressive and non-aggressive diseases [4]. As such, although G84E can be incorporated into genetic testing to enable targeted screening and family counseling [24], it currently does not have prognostic or treatment implications. In contrast, given the clinical relevance of the X285K variant established in this real-world setting, detection of this X285K variant should be interpreted in the prognostic and treatment settings. Specifically, early escalation may be justified in prostate cancer patients carrying the X285K variant, given the association of the X285K variant with aggressive prostate cancer. We detected BRCA2 variants in 1.79% of patients across the entire tested cohort. This detection rate is substantially lower than the BRCA2 P/LP detection rate (4.74%) in a previous report [15], likely reflecting the changed guidelines and inclusion of relatively lower-risk patients following that publication. Our real-world genetic data analysis was limited by a lack of a control population, a lack of data on the age of diagnosis, and often incomplete capture of clinical, pathological, and treatment data.

Our detailed functional studies uncovered a gain of function in E2F/MYC signature specific to the X285K variant. BRCA2 and HOXB13 are among the most important hereditary prostate cancer predisposition genes. Unlike many “loss-of-function” BRCA2 variants with established diagnostic, prognostic, and treatment implications, evidence supporting the role of HOXB13 variants is lacking, especially with respect to their functional characteristics, though a few studies focused on functional aspects of G84E [25, 26]. Pathogenic and likely pathogenic germline variants in BRCA2 are loss-of-function (LOF) variants caused by frameshift, premature stop, and splice site mutations that lead to protein truncation. These LOF variants explain the risk of developing cancer and are consistent with a tumor suppressor mechanism. They fit the Knudsen “two-hit” hypothesis because most tumors also have a second, somatic LOF alteration. In contrast, HOXB13 germline variants associated with the risk for prostate cancer do not exhibit protein truncation, and somatic LOF has not been demonstrated. Given the role of WT HOXB13 in the metastatic progression of prostate cancer [27-30], the findings support a gain of function in X285K with respect to its role in mediating an aggressive phenotype. The concept of gain-of-function (GOF) properties associated with a germline HOXB13 variant is entirely novel, and gain-of-function oncogenic properties of germline variants are also historically understudied [31-33]. Our findings also raise a question as to what other genomic alterations may act in concert with X285K to “activate” its oncogenic activity. Our functional study was limited by the use of a single cell line and the lack of data showing the E2F/MYC-mediated aggressive phenotypes. Further mechanistic analyses and development of this concept will require additional tools and resources (e.g., variant-specific antibodies and animal models) when they become available.

In summary, large-scale germline genetic testing results accompanied by functional laboratory experiments suggest that HOXB13 X285K is enriched in self-reported Black patients, and the X285K variant protein mediates an aggressive prostate cancer phenotype associated with gain-of-function E2F/MYC activation. Additional clinical and laboratory studies are needed to determine the functional dependency and therapeutic vulnerability of X285K tumors to specific treatments. Nevertheless, these findings underscore the clinical utility of germline genetic testing in patients with prostate cancer and provide new information to facilitate clinical interpretation of genetic testing findings in a population disproportionately affected by excess prostate cancer mortality. Our studies lend evidence to the pathogenicity of the X285K variant, which may factor in the variant classification framework to move the clinical classification of this variant from ‘likely benign” to “likely pathogenic”. These findings also motivate the identification and development of effective treatments for prostate cancer patients carrying this ancestry-specific mutation.

### REFERENCES

1. Doan D K, Schmidt K T, Chau C H, Figg W D. Germline genetics of prostate cancer: prevalence of risk variants and clinical implications for disease management. Cancers. 2021; 13:2154.
2 Giri V N, Morgan T M, Morris D S, Berchuck J E, Hyatt C, Taplin M E. Genetic testing in prostate cancer management: Considerations informing primary care. C A: A Cancer Journal for Clinicians. 2022; 72:360-71.
3. Pritchard C C, Offit K, Nelson P S. DNA-Repair Gene Mutations in Metastatic Prostate Cancer. The New England journal of medicine. 2016; 375:1804-5.
4. Isaacs W B, Cooney K A, Xu J. Updated insights into genetic contribution to prostate cancer predisposition: focus on HOXB13. Can J Urol. 2019; 26:12-3.
5 Li B, Huang Q, Wei G-H. The role of HOX transcription factors in cancer predisposition and progression. Cancers. 2019; 11:528.
6. Ewing C M, Ray A M, Lange E M, Zuhlke K A, Robbins C M, Tembe W D, et al. Germline mutations in HOXB13 and prostate-cancer risk. New England Journal of Medicine. 2012; 366:141-9.
7. Hayano T, Matsui H, Nakaoka H, Ohtake N, Hosomichi K, Suzuki K, et al. Germline variants of prostate cancer in Japanese families. PLOS One. 2016; 11: e0164233.
8. Lin X, Qu L, Chen Z, Xu C, Ye D, Shao Q, et al. A novel germline mutation in HOXB13 is associated with prostate cancer risk in Chinese men. The Prostate. 2013; 73:169-75.
9 Pomerantz M M, Li F, Takeda D Y, Lenci R, Chonkar A, Chabot M, et al. The androgen receptor cistrome is extensively reprogrammed in human prostate tumorigenesis. Nature genetics. 2015; 47:1346-51.
10. Jung C, Kim R-S, Zhang H-J, Lee S-J, Jeng M-H. HOXB13 induces growth suppression of prostate cancer cells as a repressor of hormone-activated androgen receptor signaling. Cancer research. 2004; 64:9185-92.
11. Norris J D, Chang C-Y, Wittmann B M, Kunder R S, Cui H, Fan D, et al. The homeodomain protein HOXB13 regulates the cellular response to androgens. Molecular cell. 2009; 36:405-16.
12. Darst B F, Hughley R, Pfennig A, Hazra U, Fan C, Wan P, et al. A rare germline HOXB13 variant contributes to risk of prostate cancer in men of African ancestry. European urology. 2022; 81:458-62.
13. Marlin R, Creoff M, Merle S, Jean-Marie-Flore M, Rose M, Malsa S, et al. Mutation HOXB13 c. 853delT in Martinican prostate cancer patients. The Prostate. 2020; 80:463-70.
14. Na R, Wei J, Sample C J, Gielzak M, Choi S, Cooney K A, et al. The HOXB13 variant X285K is associated with clinical significance and early age at diagnosis in African American prostate cancer patients. British journal of cancer. 2022; 126:791-6.
15. Nicolosi P, Ledet E, Yang S, Michalski S, Freschi B, O'Leary E, et al. Prevalence of germline variants in prostate cancer and implications for current genetic testing guidelines. JAMA oncology. 2019; 5:523-8.
16. Nykamp K, Anderson M, Powers M, Garcia J, Herrera B, Ho Y-Y, et al. Sherloc: a comprehensive refinement of the ACMG-AMP variant classification criteria. Genetics in Medicine. 2017; 19:1105-17.
17. Hu R, Lu C, Mostaghel E A, Yegnasubramanian S, Gurel M, Tannahill C, et al. Distinct Transcriptional Programs Mediated by the Ligand-Dependent Full-Length Androgen Receptor and Its Splice Variants in Castration-Resistant Prostate CancerTranscriptional Programs Mediated by A R-V. Cancer research. 2012; 72:3457-62.
18. Chen Z, Wu D, Thomas-Ahner J M, Lu C, Zhao P, Zhang Q, et al. Diverse A R-V7 cistromes in castration-resistant prostate cancer are governed by HoxB13. Proceedings of the National Academy of Sciences. 2018; 115:6810-5.
19. Guo H, Wu Y, Nouri M, Spisak S, Russo J W, Sowalsky A G, et al. Androgen receptor and MYC equilibration centralizes on developmental super-enhancer. Nature communications. 2021; 12:7308.
20. Islami F, Siegel R L, Jemal A. The changing landscape of cancer in the USA-opportunities for advancing prevention and treatment. Nature Reviews Clinical Oncology. 2020; 17:631-49.
21. Nelson W G, Brawley O W, Isaacs W B, Platz E A, Yegnasubramanian S, Sfanos K S, et al. Health inequity drives disease biology to create disparities in prostate cancer outcomes. The Journal of clinical investigation. 2022; 132.
22. Stopsack K H, Nandakumar S, Arora K, Nguyen B, Vasselman S E, Nweji B, et al. Differences in Prostate Cancer Genomes by Self-Reported Race: Contributions of Genetic Ancestry, Modifiable Cancer Risk Factors, and Clinical FactorsRacial Differences in Prostate Cancer Genomes. Clinical Cancer Research. 2022; 28:318-26.
23. Conti D V, Darst B F, Moss L C, Saunders E J, Sheng X, Chou A, et al. Trans-ancestry genome-wide association meta-analysis of prostate cancer identifies new susceptibility loci and informs genetic risk prediction. Nature genetics. 2021; 53:65-75.
24. Shi Z, Platz E A, Wei J, Na R, Fantus R J, Wang C-H, et al. Performance of three inherited risk measures for predicting prostate cancer incidence and mortality: a population-based prospective analysis. European Urology. 2021; 79:419-26.
25. Lu X, Fong K-w, Gritsina G, Wang F, Baca S C, Brea L T, et al. HOXB13 suppresses de novo lipogenesis through HDAC3-mediated epigenetic reprogramming in prostate cancer. Nature genetics. 2022; 54:670-83.
26. Cardoso M, Maia S, Paulo P, Teixeira M R. Oncogenic mechanisms of HOXB13 missense mutations in prostate carcinogenesis. Oncoscience. 2016; 3:288.
27. Weiner A B, Faisal F A, Davicioni E, Karnes R J, Vander Griend D J, Lotan T L, et al. Somatic HOXB13 expression correlates with metastatic progression in men with localized prostate cancer following radical prostatectomy. European Urology Oncology. 2021; 4:955-62.
28. Nerlakanti N, Yao J, Nguyen D T, Patel A K, Eroshkin A M, Lawrence H R, et al. Targeting the BRD4-HOXB13 coregulated transcriptional networks with bromodomain-kinase inhibitors to suppress metastatic castration-resistant prostate cancer. Molecular cancer therapeutics. 2018; 17:2796-810.
29. Nguyen D T, Yang W, Renganathan A, Weimholt C, Angappulige D H, Nguyen T, et al. Acetylated HOXB13 Regulated Super Enhancer Genes Define Therapeutic Vulnerabilities of Castration-Resistant Prostate Cancer. Clinical Cancer Research. 2022; 28:4131-45.
30. Yao J, Chen Y, Nguyen D T, Thompson Z J, Eroshkin A M, Nerlakanti N, et al. The homeobox gene, HOXB13, regulates a mitotic protein-kinase interaction network in metastatic prostate cancers. Scientific reports. 2019; 9:1-18.
31. Carter H, Marty R, Hofree M, Gross A M, Jensen J, Fisch K M, et al. Interaction landscape of inherited polymorphisms with somatic events in cancer. Cancer discovery. 2017; 7:410-23.
32. Porta-Pardo E, Valencia A, Godzik A. Understanding oncogenicity of cancer driver genes and mutations in the cancer genomics era. FEBS letters. 2020; 594:4233-46.
33. Farashi S, Kryza T, Clements J, Batra J. Post-GWAS in prostate cancer: from genetic association to biological contribution. Nature Reviews Cancer. 2019; 19:46-59.

### Supplementary Materials and Methods

### Analysis of Genetic Testing Data.

De-identified patient clinical data from test requisition forms were reviewed for 21,393 patients with prostate carcinoma who were referred for germline genetic testing via one commercial laboratory between 2019 and 2022. Clinical germline genetic testing was performed at Invitae (San Francisco, CA), a CLIA-certified and College of American Pathologists (CAP)-accredited diagnostic laboratory. There was a median of 84 (range 2-156) genes analyzed, with the number of genes ordered being at the discretion of the ordering clinician. Patient data were de-identified consistent with WCG-IRB approved protocol number 1167406, which waived the requirement to obtain written patient informed consent. Ethnicity data were collected for most patients by self-report at the time of test ordering.

Genomic DNA was extracted from whole blood or saliva using a QiaSymphony (Qiagen, Hilden, Germany). Targeted genes were captured using Agilent (Santa Clara, CA) SureSelect probes or Integrated DNA Technologies (Coral, IL) xGen Lockdown probes at positions where SureSelect yield was inadequate. Full gene next-generation sequencing (NGS), including+10-20 base pairs at each exon-intron boundary and targeted deep intronic variants, was performed on the Illumina (San Diego, CA) MiSeq or HiSeq 2500 to at least 450x average coverage of 2×150 reads, with a minimum of 50× required at every targeted position. Stringent process controls were used to minimize read-depth variability, and up to eight anonymous blood samples were used as control specimens in each run to measure remaining coverage variability.

Reads were aligned to the reference human genome sequence GRCh37 using Novoalign (Novocraft Technologies, Selangor, Malaysia). Sequence variants were then analyzed for indels and single-nucleotide variants (SNVs) using the Genome Analysis Toolkit (GATK). Copy-number variants were called using read-depth analysis with a proprietary Invitae database. Split-read analysis was performed as described previously to detect genomic structural variants. Identified variants were interrogated using refined American College of Medical Genetics and genomics criteria (Sherloc) and classified as pathogenic (P) or likely pathogenic (LP) if they involved large genomic events or conferred a truncating, initiation codon or splice donor/acceptor effect; if functional data showed an impact on protein function; or if pathogenicity was otherwise reported in published literature. P/LP variants were orthogonally confirmed using Sanger sequencing, multiplex ligation-dependent probe amplification (MLPA), or other appropriate orthogonal methods in accordance with Invitae standard operating practices. In this study, “pathogenic” variants were defined as those classified as P/LP.

### Statistical Analysis.

For the Prostate Cancer DHPC program analysis, exact test for contingency tables was used to compare race/ethnicity distribution between men with and without the variant, and compare patient characteristics including race/ethnicity, Gleason score, and stage, among the carriers of HOXB13 X285K, G84E and BRCA2. Patients with unknown or missing Gleason score and stage information were excluded from the analysis. Statistical analyses were completed using R (version 4.1.2) [1] and a significance threshold of α<0.05 was used. As for the estimation of population attributable risk (PAR), first, single variant carrier frequency data for HOXB13 X285K in black population and G84E in white population were extracted from male subjects in the Genome Aggregation Database (gnomAD). The carrier frequency for BRCA2 was the sum of carrier frequencies for 247 BRCA2 P/LP variants detected in the entire population. The prevalence of HOXB13 X285K in the black subject with prostate cancer, G84E in white population with prostate cancer, and BRCA2 in prostate cancer is estimated from the samples in DETECT Program. PAR was calculated by 1−((1-proportion of variant+among men with prostate cancer)/(1−proportion of variant+ in the population)). The statistical analyses for in vitro assays were performed using GraphPad Prism ver. 9. For multiple comparisons, groups were first compared with one-way ANOVA. If there were statistical differences among groups as determined by ANOVA (p<0.05), Turkey's multiple comparison tests were performed for pairwise comparison.

### X285K Protein Structure.

The HOXB13 (X285K) protein structure was predicted by AlphaFold v2.1.0 [2] and visualized by the NCBI protein viewer.

### Cell Culture.

LNCaP95 is an androgen-independent prostate cancer cell line derived from the parental LNCaP [3]. LNCaP95 was cultured in RPMI 1640 media without phenol red (Thermo Fisher Scientific, #11835055) supplemented with 10% charcoal-stripped FBS (Sigma, #F6765). Synthetic androgen methyltrienolone (R1881) was purchased from PerkinElmer (#NLP0050). Cell cultures are routinely authenticated and tested to rule out mycoplasma contamination.

### Generation of Tet-ON Inducible Stable Lines.

Vector construction: Tet-ON 3G bidirectional inducible expression system (Clontech, #631337) was used to generate stable lines. Prior to ligation, inserts and donor vectors with sticky ends were blunted by a Quick blunting kit (NEB, #E1201L). For HOXB13 knockdown, SMARTvectors carrying RFP-tagged HOXB13 shRNA (catalog #V3SH11252-224939824, clone I.D. #V3IHSHER_4844474) and control non-targeting shRNA (catalog #VSC6573) were purchased from Dharmacon. Fragments containing RFP-tagged HOXB13 shRNA or non-targeting shRNA flanked by SMARTvector universal scaffolds were excised from original vectors with SnaBI and ClaI and were inserted into multi-cloning site 1 of the pTRE3G-BI vector at EcoRV site. For HOXB13 expression, cDNA open reading frame (ORF) of the HOXB13 gene was purchased from ORIGENE (catalog #RC209991). For wild-type (WT) HOXB13 expression, HOXB13 ORF with flag tag was excised from an original vector with Smal and AsiSI and inserted into multi-cloning site 2 of the pTRE3G-BI vector at Smal site. For X285K expression, HOXB13 3′UTR fragment was first amplified from human genomic DNA using the following primers: forward: 5′-GATTACCATCTGGTTTCAGAACCGC-3′ (SEQ ID NO:1); reverse: 5′-GCTCAATTCATGAAAGCGGTTTCTAAAG-3′ (SEQ ID NO:2). This amplified fragment was digested with AfeI and Eco53kI and inserted into AfeI-and-MluI-cut ORIGENE WT HOXB13 vector. To generate one-nucleotide deletion at the HOXB13 stop codon (c.853delT) leading to a frameshift (stop codon replaced by lysine: AAG), c.853delT was introduced to the above vector (ORIGENE WT HOXB13 vector carrying HOXB13 3′UTR fragment) by site-directed mutagenesis using the following primers; forward: 5′-GCGOTACCCCTAAGAGATCTCCTTGCCTGGG-3′ (SEQ ID NO:3); reverse: 5′-GGAGATCTCTTAGGGGTAGCGCTGTTCTTCA-3′ (SEQ ID NO:4). This X285K fragment was subsequently excised with Smal and AsiSI and inserted into multi-cloning site 2 of pTRE3G-BI vector (carrying RFP-tagged HOXB13 shRNA at multi-cloning site 1) at Smal site.

Establishment of 11 stable lines.

HOXB13 inducible stable lines were established in accordance with the manufacturer's protocol. First, clones stably expressing rtTA (reverse tetracycline-controlled transactivator) were isolated, and a clone expressing the highest level of rtTA determined by qRT-PCR was subjected to the second transfection with the pTRE3G-BI vectors carrying WT or X285K HOXB13 and RFP-tagged HOXB13 shRNA. Stable clones were selected with 0.25-0.5 μg/mL puromycin, and clones that express HOXB13 and homogeneous RFP expression upon Dox treatment were selected, leading to five WT clones and three X285K clones. Three NT clones were generated on the basis of homogeneous RFP upon Dox treatment. To induce exogenous gene expression, 1-1000 ng/mL Dox was used to test dose dependency (not shown) and narrowed down to the range of 0.01-20 ng/mL to tightly regulate the gene expression level as well as to minimize the toxic effects of Dox, although the recommended concentration of Dox is 100-1000 ng/mL according to manufacturer's protocol.

Sample Preparation for qRT-PCR and RNA Sequencing.

Cells were treated with indicated concentrations of doxycycline (Dox, Sigma, #D9891) for 72 hours. At the 72-hour timepoint, 1 nM R1881 was added (for R1881-untreated samples, an equal volume of ethanol was added) and cells were incubated for an additional 24 hours. At 96-hour timepoint, cells were lysed in TRIzol reagent (Invitrogen #15596018) (Supplementary FIG. 3A). Total RNA was extracted using RNeasy mini kit (QIAGEN #74104) and quantified with Nanodrop.

qRT-PCR Analysis.

Each of the 11 clones was subjected to 4 experimental conditions (+/−Dox, +/−R1881) as shown in Supplementary FIG. 3A. Total RNA was extracted using RNeasy mini kit (QIAGEN #74104), followed by cDNA synthesis with SuperScript IV first-strand synthesis system (Thermo Fisher Scientific, #18091050). The iQ SYBR Green Supermix (BioRad, #1708882) was used for qRT-PCR with the following primer sets: HOXB13 forward: 5′-GAACAGCCAGATGTGTTGCCAG-3′ (SEQ ID NO:5); HOXB13 reverse: 5′-GGAATGCGTTTCTTGCGGCC-3′ (SEQ ID NO:6); CCNB1 forward: 5′-CTGAGCCAGAACCTGAGCCTG-3′ (SEQ ID NO:7); CCNB1 reverse: 5′-GTCTTCTTCTGCAGGGGCACATC-3 ‘(SEQ ID NO:8); GAPDH forward: 5’-AGCACCAGGTGGTCTCCTC-3′ (SEQ ID NO:9); GAPDH reverse: 5′-CCCTGTTGCTGTAGCCAAATTC-3′ (SEQ ID NO:10). Changes in gene expression were compared by the comparative Ct method. LNCaP95 stably expressing rtTA (reverse tetracycline-controlled transactivator) without any treatments was used as a reference sample. To ensure reproducibility of CCNB1 measurements across the 11 clones and four treatments (leading to 44 RNA samples), we adopted the “nested-split” approach to account for variations that may be introduced during the entire process (cell culture, treatment, RNA extraction, generation of cDNA, PCR). The first split occurred at the cell culture level (i.e., two culture wells replicating the entire process for cell treatment and downstream steps), and the second split at the cDNA level (i.e., two PCR reactions for the same cDNA). This “nested-split” process generates four measurements for each of the 44 samples. These 4 measurements were treated as technical replicates.

### Western Blot Analysis.

Cells were treated with indicated concentrations of doxycycline (Dox, Sigma, #D9891) for 72 hours. At the 72-hour timepoint, 1 nM R1881 was added (for R1881-untreated samples, an equal volume of ethanol was added) and cells were incubated for an additional 24 hours. At the 96-hour timepoint, cells were washed with PBS and lysed in buffer containing 1× passive lysis buffer (Promega, #E1941), protease inhibitor (Sigma, #11873580001), and phosphatase inhibitor (Sigma, #490684500). Protein concentrations were determined using BCA assay (Thermo Fisher Scientific, #23227) and 20 μg of protein was separated on 4-15% SDS-PAGE gel (Bio-Rad, #1610772). Proteins were transferred to PVDF membranes followed by incubation with the following primary antibodies: c-Myc (CST, #9402), HOXB13 (CST, #90944), Cyclin B1 (CST, #4138) and β-actin (Sigma, #A2228). Signals were detected by enhanced chemiluminescence (Thermo Fisher Scientific, #34580).

### RNA Sequencing and Differential Gene Expression Analysis.

Each of the 11 clones was subjected to 4 experimental conditions (+/−Dox, +/−R1881) as shown in Supplementary FIG. 3A, generating 44 samples for RNA-Seq. The RNA-Seq library was prepared using Illumina TruSeq stranded mRNA sample preparation kit (Illumina, San Diego, CA) and sequencing was performed using 100 bp paired-end sequencing module with Illumina HiSeq 3000 sequencing platform. Alignment was performed with HiSAT2 algorithm [4] using UCSC human genome build hg19. Gene expression quantification was performed with StringTie [5] using gencode v24. Read count was assigned to each gene using prepDE.py provided by StringTie. Differential gene expression analysis was performed in edge [6]. Differentially expressed genes (DEGs) were defined by 1) fold change >2; 2) multiple-test adjusted p-value <0.05; and 3) average FPKM>5 (per comparison group). For comparisons cross treatments, edgeR was configured for paired sample comparison (by clonal expansion ID). For comparison across mutation types, group-wise comparison was used. RNA-Seq data was deposited in the GEO database with a GEO accession number (GSE240364).

### RNA-Seq Data Functional Analysis.

Heatmap of each comparison were generated using pHeatmap function (Bioconductor). For Gene set enrichment analysis, the Bioconductor package fgsea [7], a fast implementation to the original GSEA algorithm, was used. Fold change was used to generate a rank list for GSEA after removing 75% of genes with low expression level in both comparison groups. This filtration removed most weakly expressed non-coding transcripts and retained ~15K genes as there are 60,308 genes in gencode annotation V24.

### HOXB13 Chromatin Immunoprecipitation Sequencing by Active Motif.

Representative Tet-ON LN95 clones expressing similar levels of exogenous WT or X285K HOXB13 (LN95WT clone 26 and LN95X285K clone 21) were chosen and treated in the same way as RNA-seq samples as shown in Supplementary FIG. 3A. Cells were fixed according to Active Motif online protocol. HOXB13 antibody (CST, #90944) was used for ChIP-Seq. ChIP-Seq was completed by Active Motif according to their proprietary methods. The 75-nt single-end (SE75) sequence reads generated by Illumina sequencing are mapped to the genome using the BWA algorithm (“bwa aln/samse” with default settings). Alignment information for each read is stored in the BAM format. Only reads that pass Illumina's purity filter, align with no more than 2 mismatches, and map uniquely to the genome are used in the subsequent analysis. Mapped reads (to hg38 genome build) of 45.6M and 41.6M reads were obtained from LN95WT (Dox+/R1881+) and LN95X285K (Dox+/R1881+) pull-downs, respectively. Peaks were annotated with their nearest genes (within-10K upstream to 1K downstream of the transcription start site (TSS)). Standard Normalization was done by random sampling. The tag number of all samples (within a comparison group) was reduced by random sampling to the number of tags present in the smallest sample. Normalized peak results were visualized by IGV genome browser. Peaks Differential peak analysis was performed using DiffBind package (R/Bioconductor).

### REFERENCES

1. Team R C. R: A language and environment for statistical computing. R Foundation for Statistical Computing, Vienna, Austria. R-project. org/2016
2 Jumper J, Evans R, Pritzel A, et al. Highly accurate protein structure prediction with AlphaFold. Nature 2021; 596 (7873): 583-89
3. Hu R, Lu C, Mostaghel E A, et al. Distinct Transcriptional Programs Mediated by the Ligand-Dependent Full-Length Androgen Receptor and Its Splice Variants in Castration-Resistant Prostate CancerTranscriptional Programs Mediated by AR-V. Cancer research 2012; 72 (14): 3457-62
4. Kim D, Langmead B, Salzberg S L. HISAT: a fast spliced aligner with low memory requirements. Nature methods 2015; 12 (4): 357-60
5. Pertea M, Kim D, Pertea G M, Leek J T, Salzberg S L. Transcript-level expression analysis of RNA-seq experiments with HISAT, StringTie and Ballgown. Nature protocols 2016; 11 (9): 1650-67
6. Robinson M D, McCarthy D J, Smyth G K. edgeR: a Bioconductor package for differential expression analysis of digital gene expression data. bioinformatics 2010; 26 (1): 139-
7. Korotkevich G, Sukhov V, Budin N, Shpak B, Artyomov M N, Sergushichev A. Fast gene set enrichment analysis. BioRxiv 2016:060012

TABLE 6
Summary of germline genetic testing results from the Prostate Cancer DHPC program.

| | HOXB13 | | HOXB13 | | BRCA2 | | p-value for |
|---|---|---|---|---|---|---|---|
| | p.X285K | | p.G84E | | P/LP variants | | difference |
| N variants detected/N evaluated (%) | 38/21,091 | (0.18%) | 173/21,091 | (0.82%) | 382/21,362 | (1.79%) | |
| Black | 27/2,680 | (1.01%) | 3/2,680 | (0.11%) | 52/2,715 | (1.92%) | |
| White | 1/13,628 | (0.01%) | 153/13,628 | (1.12%) | 218/13,821 | (1.58%) | |
| Other/Multiracial | 10/4,783 | (0.21%) | 17/4,783 | (0.36%) | 112/4,826 | (2.32%) | |
| | p = 2.2 × 10⁻¹⁶* | | p = 1.8 × 10⁻¹²* | | p = 0.0037* | | |
| Race/ethnicity in variant + patients | | | | | | | p = 2.2 × 10⁻¹⁶ ** |
| Black | 27 | (71.1%) | 3 | (1.7%) | 52 | (13.6%) | |
| White | 1 | (2.6%) | 153 | (88.4%) | 218 | (57.1%) | |
| Other/Multiracial | 10 | (26.3%) | 17 | (9.8%) | 112 | (29.3%) | |
| Gleason score | | | | | | | p = 2.8 × 10⁻⁶ ** |
| ≤6 | 1 | (3.1%) | 18 | (11.7%) | 12 | (3.7%) | |
| 7 | 11 | (34.4%) | 60 | (39.0%) | 86 | (26.3%) | |
| 8 | 13 | (40.6%) | 31 | (20.1%) | 67 | (20.5%) | |
| 9-10 | 7 | (21.9%) | 45 | (29.2%) | 162 | (49.5%) | |
| unknown | 6 | | 19 | | 55 | | |
| Stage | | | | | | | p = 6.6 × 10⁻⁴ ** |
| Stage II | 1 | (6.3%) | 14 | (14.6%) | 10 | (3.9%) | |
| Stage III | 0 | (0.0%) | 17 | (17.7%) | 27 | (10.5%) | |
| Stage IV | 15 | (93.7%) | 65 | (67.7%) | 221 | (85.6%) | |

| | | | |
|---|---|---|---|
| unknown | 22 | 77 | 124 |

*Comparison of race/ethnicity distribution between patients with and without the variant;
** Comparison among the three variants. All p-values calculated with exact test. Patients with unknown or missing Gleason score and stage information were excluded from the analysis.

TABLE 7
Estimation of population attributable risk
(PAR) for HOXB13 and BRCA2 variants

| | DETECT Program | gnomAD* | PAR** |
|---|---|---|---|
| HOXB13 X285K (Black) | 0.010075 | 0.003924 | 0.62% |
| HOXB13 G84E (White) | 0.011227 | 0.005317 | 0.59% |
| BRCA2 (entire population) | 0.017882 | 0.006034 | 1.19% |

*Single variant carrier frequency data for HOXB13 X285K and G84E were from male subjects in the Genome Aggregation Database (gnomAD). The carrier frequency for BRCA2 was the sum of carrier frequencies for 247 BRCA2 P/LP variants detected in the entire population.
**Population attributable risk (PAR) was calculated by 1 − ((1 − proportion of variant + among men with prostate cancer)/(1 − proportion of variant + in the population))

TABLE 8
Gleason score and stage by variant status for self-reported Black patients only

| | HOXB13/BRCA | | HOXB13 | | p- | HOXB13 | | p- | BRCA2 | | p- | BRCA1 | | p- |
|---|---|---|---|---|---|---|---|---|---|---|---|---|---|---|
| | negative | | c.853del | | value* | G84E | | value* | P/LP | | value* | P/LP | | value* |
| Gleason score | | | | | | | | | | | | | | |
| ≤6 | 188 | (8.8%) | 1 | (4.5%) | p = 0.03 | 0 | (0%) | p = 0.74 | 2 | (4.5%) | p = 0.57 | 1 | (14.3%) | p = 0.31 |
| 7 | 833 | (39.0%) | 7 | (31.8%) | | 2 | (100%) | | 15 | (34.1%) | | 1 | (14.3%) | |
| 8 | 399 | (18.7%) | 10 | (45.5%) | | 0 | (0%) | | 8 | (18.2%) | | 1 | (14.3%) | |
| 9-10 | 718 | (33.6%) | 4 | (18.2%) | | 0 | (0%) | | 19 | (43.2%) | | 4 | (57.1%) | |
| unknown | 488 | | 5 | | | 1 | | | 8 | | | 0 | | |
| Stage | | | | | | | | | | | | | | |
| Stage II | 223 | (15.3%) | 1 | (8.3%) | p = 0.66 | 0 | (0%) | p = 1 | 2 | (6.3%) | p = 0.29 | 1 | (50.0%) | p = 0.41 |
| Stage III | 111 | (7.6%) | 0 | (0.0%) | | 0 | (0%) | | 1 | (3.1%) | | 0 | (0.0%) | |
| Stage IV | 1127 | (77.1%) | 11 | (91.7%) | | 0 | (0%) | | 29 | (90.6%) | | 1 | (50.0%) | |

| | | | | | |
|---|---|---|---|---|---|
| unknown | 1,165 | 15 | 3 | 20 | 5 |

*Comparison between two groups (each variant positive group vs. control group negative for the named HOXB13/BRCA variants). All p-values were calculated using exact test for contingency table. Patients with unknown or missing Gleason score and stage information were excluded from the analysis.

1. A method for identifying a subject as having an increased risk of prostate cancer (PCa) comprising the step of detecting single nucleotide polymorphism rs77179853 in DNA isolated from the subject, wherein the presence of the rs77179853 SNP identifies the subject as having an increased risk of aggressive prostate cancer.

2. The method of claim 1, wherein the PCa is aggressive PCa.

3. The method of claim 2, wherein the subject is of African descent.

4. The method of claim 3, wherein, the subject has a family history of PCa.

5. The method of claim 1, wherein the subject is identified as having an increased risk of PCa at an early age.

6. The method of claim 1, wherein the DNA is isolated from a biological sample selected from the group consisting of prostate tissue biopsy, fresh or archival surgical specimen, saliva, urine and blood.

7. The method of claim 1, further comprising the step of administering a treatment of prostatectomy, radiation, chemotherapy, immunotherapy or a combination thereof to the subject having an increased risk of PCa.

8. The method of claim 1, wherein the detecting step comprises nucleic acid amplification.

9. The method of claim 1 wherein the detecting step comprises a hybridization reaction.

10. The method of claim 9, wherein the hybridization reaction further comprises hybridizing the sample to one or more primer sets.

11. The method of claim 10, wherein the hybridization reaction is a polymerase chain reaction (PCR).

12. The method of claim 11, wherein the PCR is reverse transcription PCR.

13. The method of claim 1, wherein the detecting step comprises a microarray or DNA sequencing.

14. A method for determining whether a subject will not respond to androgen or androgen receptor-directed therapies comprising the step of detecting single nucleotide polymorphism rs77179853 in DNA isolated from the subject, wherein the presence of the rs77179853 SNP indicates that the subject will not respond, or have a poor outcome, to androgen or androgen receptor-directed therapies.

15. A method for treating a subject at risk of or having PCa comprising the steps of:
(a) detecting single nucleotide polymorphism rs77179853 in DNA isolated from the subject, wherein the presence of the rs77179853 SNP identifies the subject as having poor response to androgen or androgen receptor-directed therapies; and
(b) administering a non-androgen or non-androgen receptor therapies to treate the subject.

16. The method of claim 15, wherein the non-androgen or non-androgen receptor therapies treatment comprises prostatectomy, radiation, chemotherapy, immunotherapy or a combination thereof.

17. A method for identifying a subject as having an increased risk of prostate cancer (PCa) comprising the step of detecting a protein comprising SEQ ID NO:13 or a nucleic acid encoding SEQ ID NO: 13 in a sample obtained from the subject, wherein the presence of the protein comprising SEQ ID NO: 13 or the nucleic acid encoding SEQ ID NO:13 identifies the subject as having an increased risk of aggressive prostate cancer.

18. A method for determining whether a subject will not respond to androgen therapy comprising the step of detecting a protein comprising SEQ ID NO:13 or a nucleic acid encoding SEQ ID NO: 13 in a sample obtained from the subject, wherein the presence of the protein comprising SEQ ID NO: 13 or the nucleic acid encoding SEQ ID NO:13 indicates that the subject will not respond to androgen therapy.

19. The method of claim 18, wherein amino acid 285 of SEQ ID NO: 13 is a lysine.

20. The methods of claim 18, wherein detecting a protein comprising SEQ ID NO:13 or a nucleic acid encoding SEQ ID NO:13 comprises detecting one or more of amino acids 285-380 of SEQ ID NO:13.

21. The methods of claim 18, wherein detecting a protein comprising SEQ ID NO:13 or a nucleic acid encoding SEQ ID NO:13 comprises detecting one or more of amino acids 344-364 of SEQ ID NO: 13.

22. The method of claim 18, wherein the subject is of African descent.
